(19) United States
(12) Patent Application Publication
(10) Pub. No.: US 2025/0009744 A1
(43) Pub. Date: Jan. 9, 2025

(54) USE OF METABOLIC REGULATORS FOR THE TREATMENT OF LIPOTOXICITY IN THE LUNGS

(71) Applicant: YISSUM RESEARCH DEVELOPMENT COMPANY OF THE HEBREW UNIVERSITY OF JERUSALEM LTD., Jerusalem (IL)

(72) Inventors: Yaakov NAHMIAS, Mevaseret Zion (IL); Avner EHRLICH, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/379,033

(22) Filed: Oct. 11, 2023

Related U.S. Application Data
(63) Continuation of application No. PCT/IL2022/050392, filed on Apr. 13, 2022.
(60) Provisional application No. 63/174,180, filed on Apr. 13, 2021.

(51) Int. Cl. A61K 31/505; A61K 31/201; A61K 31/216; A61P 11/00

(52) U.S. Cl. CPC A61K 31/505; A61K 31/201; A61K 31/216; A61P 11/00

(57) ABSTRACT

Methods of treating lipotoxicity in the lungs by administering a metabolic regulator are provided.

### CROSS-REFERENCE TO RELATED APPLICATIONS

This application is a Bypass Continuation of PCT Patent Application No. PCT/IL2022/050392 having International filing date of Apr. 13, 2022, which claims the benefit of priority of U.S. Provisional Patent Application No. 63/174,180 filed Apr. 13, 2021, the contents of which are all incorporated herein by reference in their entirety.

### REFERENCE TO AN ELECTRONIC SEQUENCE LISTING

The contents of the electronic sequence listing (HUJI-P-073-US.xml; Size: 36,027 bytes; and Date of Creation: Oct. 11, 2023) is herein incorporated by reference in its entirety.

### FIELD OF INVENTION

The present invention is in the field of treatment of lipotoxicity.

### BACKGROUND OF THE INVENTION

Lipotoxicity is a syndrome that results from accumulation of phospholipid molecules and lipid intermediates in non-adipose tissue. This accumulation, if it is sufficiently large or long lasting can result in cellular dysfunction and death. Lipotoxicity has been studied in the kidneys, liver, heart, skeletal muscle and even the brain. It is believed to play a role in heart failure, obesity, diabetes as well as neurodegenerative disease.

In lipotoxic cells there is an imbalance between the production of lipids (synthesis and intake) and the use of lipids (catabolismoxidation and export). The resultant toxicity manifests itself differently depending on the organ in which the lipotoxicity occurs. This toxicity can cause a wide range of symptoms. As lipotoxicity manifests itself differently in different organs, an organ specific treatment modality is often required. Methods and compositions for treating lipotoxicity in the lungs, which heretofore was not an identified form of lipotoxicity, are greatly needed.

### SUMMARY OF THE INVENTION

The present invention provides methods of treating or preventing lipotoxicity in the lungs by administering a metabolic regulator.

According to a first aspect, there is provided a method of treating lipotoxicity in a lung of a subject in need thereof, the method comprising administering to the subject a therapeutic composition comprising at least one metabolic regulatory drug, thereby treating lipotoxicity in the lungs in a subject.

According to another aspect, there is provided a therapeutic composition comprising at least one metabolic regulator for use in treating lipotoxicity in a lung of a subject in need thereof.

According to another aspect, there is provided a therapeutic composition comprising at least one metabolic regulator for use in a method of the invention.

According to some embodiments, the method is for treating a disease, condition or disorder characterized by lipotoxicity in lungs.

According to some embodiments, the lipotoxicity is characterized by a reduction in carnitine palmitoyltransferase 1A (CPT1A) in lung tissue from the subject.

According to some embodiments, the lipotoxicity is caused by a bacterial or viral infection of the lung.

According to some embodiments, the virus is an influenza virus.

According to some embodiments, the influenza is a bird or swine influenza.

According to some embodiments, the influenza is selected from H1N1, H3N2 and H5N1 influenza.

According to some embodiments, the bacterial or viral infection produces pneumonia in the lung.

According to some embodiments, the viral infection is not a coronavirus infection.

According to some embodiments, the disease is selected from influenza, idiopathic pulmonary fibrosis (IPF), chronic obstructive pulmonary disease (COPD), infant respiratory distress syndrome (IRDS), acute respiratory distress syndrome (ARDS/RDS), acute lung injury (ALI), vaping-associated lung injury, pulmonary alveolar proteinosis (PAP), pneumonia and tuberculosis (TB).

According to some embodiments, the disease is selected from influenza, IPF and COPD.

According to some embodiments, the method further comprises confirming lipotoxicity in the lung of the subject before administering the therapeutic composition.

According to some embodiments, the confirming comprises confirming inhibition of a peroxisome proliferator-activated receptor (PPAR) pathway or reduced expression of a gene in the PPAR pathway.

According to some embodiments, the PPAR is PPAR alpha (PPARA).

According to some embodiments, the confirming comprises confirming reduced expression of CPT1A.

According to some embodiments, the subject is a mammal, optionally wherein the mammal is a human.

According to some embodiments, the subject is not currently or was not previously treated with the metabolic regulatory drug.

According to some embodiments, the subject does not suffer from a metabolic disease or disorder treatable by the regulatory drug.

According to some embodiments, the metabolic regulatory drug is a PPAR agonist.

According to some embodiments, the PPAR agonist is a PPARA agonist.

According to some embodiments, the PPARA agonist is selected from a fibrate, pirinixic acid and conjugated linoleic acid (CLA) and derivatives thereof.

According to some embodiments, the CLA is selected from 9-CLA and 10-CLA.

According to some embodiments, the fibrate is selected from aluminum clofibrate, bezafibrate, ciprofibrate, choline fenofibrate, clinofibrate, clofibrate, clofibride, fenofibrate, gemfibrozil, ronifibrate, fenofibric acid, pemafibrate, and simfibrate.

According to some embodiments, the fibrate is fenofibrate.

According to some embodiments, the fibrate is not gemfibrozil.

According to some embodiments, the administering is oral or intravenous administering.

According to some embodiments, the metabolic regulatory drug is administered on the first day of administration at twice a dose administered for treating a metabolic condition and is subsequently administered at the dose for treating a metabolic condition.

According to some embodiments, the treating comprises at least one of reduced phospholipid accumulation in lung cells, reduced viral load, reduced symptoms, reduced inflammation, reduced risk of Acute respiratory distress syndrome (ARDS), reduced risk of a cytokine storm and reduced risk of death.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C: Table of gene enrichment analysis of RNA-Seq data isolated from Human tracheobronchial epithelial (HTBE) human lung epithelial cells infected for 18 hours with influenza strains (1A) H5N1 (bird flu), (1B) H1N1, and (1C) H3N2 (swine flu).

FIGS. 2A-B: Diagrams showing mapping of the differentially expressed genes of influenza strain H5N1 on metabolic pathways of (2A) central carbon metabolism and (2B) PPAR signaling pathway. Think black lines denote upregulated expression and grey lines denotes down regulated expression. Genes with a “+” are upregulated and genes with a “−” are downregulated.

FIG. 3: PPARα activity is inhibited by different strains of the influenza virus. Bar chart of transcripts per million of PPARα activity marker carnitine palmitoyltransferase 1A (CPT1A) in response to influenza virus infection by strains H1N1, H3N2 and H5N1. n=2; * and *** represent positive false discovery rate (pFDR; q-value)<0.05, <0.001, respectively, as determined by Sleuth.

FIG. 4: Bar chart analysis of CCL20, CXCL1, CXCL2, CXCL5, GCSF, IL-1b, IL-6, IL-8, NFKB, SAA2, and TNFα expression by qRT-PCR as markers of immunoinflammatory stress in Human tracheobronchial epithelial (HTBE) cell in response to infection with H1N1, H3N2, and H5N1 influenza virus. Influenza infection result in significant upregulation of cytokines and inflammation markers (n=2, p<0.05).

FIGS. 5A-E: (5A) Bar chart analysis of PPARα and CPT1α expression by qRT-PCR 5. PPARα knockout results in a significant decrease in lipid oxidation regulator CPT1α (n=6, p<0.001). (5B) Western blot analysis of PPARα protein in bronchial epithelial cells or PPARα CRISPR-KO bronchial epithelial cells. (5C) Microscopic analysis of lipid accumulation in PPARα CRISPR-knockout lung cells 5 days after selection. (5D) Bar chart quantification of the results in 5C. PPARα CRISPR-knockout results in 50% increase in triglycerides (n=6, p<0.05) and 60% increase in phospholipids (n=6, p<0.001). (5E) Bar chart analysis of CCL20, CXCL1, CXCL2, CXCL5, GCSF, IL-1b, IL-6, IL-8, NFKB, SAA2, and TNFα by qRT-PCR as markers of immunoinflammatory stress. KO of PPARα results in significant upregulation of cytokines and inflammation markers even only 5 days after selection (n=6, p<0.05). * p<0.05, ** p<0.01, *** p<0.001 in a 2-sided heteroscedastic student's t-test against control. Bar=40 μm. Error bars indicate S.E.M.

FIGS. 6A-C: (6A) Microscopic analysis of lipid accumulation in lung cells induced by 10 μM of PPARγ agonist rosiglitazone and 100 μM oleic acid with or without 20 μM fenofibrate for 5 days. (6B) Bar chart quantification of the results of 6A. Lipogenic induction (rosiglitazone) results in 65% increase in triglycerides (n=6, p<0.05) and 75% increase in phospholipids (n=6, p<0.001), but of which were completely reversed when treated with fenofibrate. (6C) Bar chart analysis of CCL20, CXCL1, CXCL2, CXCL5, GCSF, IL-1b, IL-6, IL-8, NFKB, SAA2, and TNFα by qRT-PCR as markers of immunoinflammatory stress. Lipogenic induction (rosiglitazone) results in significant upregulation of chemokines, cytokines, and inflammation markers, which is reversed by fenofibrate (n-6, p<0.05). * p<0.05, ** p<0.01, *** p<0.001 in a 2-sided heteroscedastic student's t-test against control. Bar=30 μm. Error bars indicate S.E.M.

FIGS. 7A-D: Bar charts analysis of expression of (7A, 7C) CPT1A and (7B, 7D) CCL20, CXCL1, CXCL2, CXCL5, CSF3, IL-1b, NFKB, SAA2, IL-6, CXCL8, and TNFα by qRT-PCR as markers of immunoinflammatory stress in (7A-B) control and IFP FFPE lung tissue and (7C-D) isolated small airway epithelial cells from control never having smoked subjects and COPD subjects.

### DETAILED DESCRIPTION OF THE INVENTION

The present invention, in some embodiments, provides methods of treating and/or preventing lipotoxicity in the lungs by administering a metabolic regulator.

This invention is based, at least in part, on the surprising finding that conditions and diseases of the lungs often comprise a lipotoxic component that contributes to the symptoms and severity of the disease. The metabolic response of primary lung epithelial cells to influenza infection was tracked, and there was observed an increase in lipid accumulation, driven in part by inhibition of lipid catabolism and specifically inhibition of the peroxisome proliferator-activated receptor (PPAR) pathway. Other lung conditions were also examined and showed the same hallmarks of lipid accumulation and toxicity. This indicates that metabolic treatments, and in particular PPAR inhibitors, are an effective treatment for lipotoxic damage of the lungs. In contrast, lung diseases not characterized by lipotoxicity, such as pulmonary hypertension, would not benefit from a metabolic treatment.

By a first aspect, there is provided a method of treating lipotoxicity of a lung of a subject in need thereof, the method comprising administering a metabolic regulatory drug to the subject, thereby treating lipotoxicity of a lung.

By another aspect, there is provided a method of preventing lipotoxicity of a lung of a subject in need thereof, the method comprising administering a metabolic regulatory drug to the subject, thereby preventing lipotoxicity of a lung.

By another aspect, there is provided a method of reducing the risk of a symptomatic lung infection in a subject in need thereof, the method comprising administering a metabolic regulatory drug to the subject, thereby reducing the risk of a symptomatic lung infection.

By another aspect, there is provided a metabolic regulatory drug for use in treating lung lipotoxicity in a subject in need thereof.

By another aspect, there is provided a metabolic regulatory drug for use in preventing lung lipotoxicity in a subject in need thereof.

By another aspect, there is provided a metabolic regulatory drug for use in reducing the risk of a symptomatic lung infection in a subject in need thereof.

By another aspect, there is provided a metabolic regulatory drug for use in a method of the invention.

In some embodiments, the method is a method of treating a disease, condition or disorder of the lungs. In some embodiments, the method is a method of treating a disease, condition or disorder characterized by lipotoxicity in the lungs. In some embodiments, the method is a method of treating a disease, condition or disorder that comprises lipotoxicity in the lungs. In some embodiments, the method is a method of treating a disease, condition or disorder that comprises at least one symptom that is caused by lipotoxicity in the lungs. In some embodiments, lipotoxicity of the lungs is lung lipotoxicity.

In some embodiments, the lipotoxicity is caused by a lung disease. In some embodiments, the lipotoxicity is caused by a lung condition. In some embodiments, the lung disease is asthma. In some embodiments, the lung disease is chronic obstructive pulmonary disease (COPD). In some embodiments, the lung disease is cystic fibrosis. In some embodiments, the lung condition is lung transplant. In some embodiments, the disease is a lung infection. In some embodiments, the infection is a bacterial infection. In some embodiments, the infection is a viral infection. In some embodiments, the infection is not a viral infection. In some embodiments, the infection is not a corona virus infection. In some embodiments, the infection is not a Severe acute respiratory syndrome coronavirus 2 (SARS-Cov-2) infection. In some embodiments, the infection is influenza. In some embodiments, the disease is pneumonia. In some embodiments, the disease is an inflammatory lung disease. In some embodiments, the lung disease is idiopathic pulmonary fibrosis (IPF). In some embodiments, the lung disease is chronic obstructive pulmonary disease (COPD). In some embodiments, the lung disease is infant respiratory distress syndrome (IRDS). In some embodiments, the lung disease is acute respiratory distress syndrome (ARDS/RDS). In some embodiments, the lung disease is acute lung injury (ALI). In some embodiments, the lung disease is vaping-associated lung injury. In some embodiments, the lung disease is pulmonary alveolar proteinosis (PAP). In some embodiments, the lung disease is tuberculosis (TB). In some embodiments, the lung disease is selected from influenza, IPF and COPD.

In some embodiments, the lipotoxicity is caused by an infection. In some embodiments, the infection is a bacterial infection. In some embodiments, the infection is a viral infection. In some embodiments, the infection is not a viral infection. In some embodiments, the infection causes pneumonia. In some embodiments, the infection is capable of causing pneumonia. In some embodiments, the virus is a pulmonary virus. In some embodiments, the virus is a respiratory virus. In some embodiments, the virus is selected from an influenza virus, a respiratory syncytial virus, a parainfluenza virus, a metapneumovirus, a rhinovirus, a coronavirus, an adenovirus and a bocavirus. In some embodiments, the virus is selected from an influenza virus, a respiratory syncytial virus, a parainfluenza virus, a metapneumovirus, a rhinovirus, an adenovirus and a bocavirus. In some embodiments, the virus is not a corona virus. In some embodiments, the virus is not SARS-Cov-2 virus. In some embodiments, the virus is an RNA virus. In some embodiments, the virus is a DNA virus.

In some embodiments, the virus is an influenza virus. In some embodiments, the influenza virus is a human influenza virus. In some embodiments, the influenza virus is a bird influenza virus. In some embodiments, a bird influenza is an avian influenza. In some embodiments, the influenza virus is a swine influenza. In some embodiments, an avian or swine influenza is a virus that originally was in a non-human species and has jumped to humans (i.e., evolved to infect humans). In some embodiments, the influenza is influenza A. In some embodiments, the influenza is influenza B. In some embodiments, the influenza is a specific strain of influenza. In some embodiments, the influenza is selected from H1N1, H3N2, H5N1, B, H7N9, H2N2, H4N6, H5N8, H6N4, H7N2, H7N3, H7N7, H8N4, H9N2, H10N3, H10N8, H11N9 and H13N6. In some embodiments, the influenza is selected from H1N1, H3N2, H5N1, B, and H7N9. In some embodiments, the influenza is selected from H1N1, H3N2, H5N1, and B. In some embodiments, the influenza is selected from H1N1, H3N2, and H5N1. In some embodiments, the influenza is H1N1. In some embodiments, the influenza is H3N2. In some embodiments, the influenza is H5N1.

In some embodiments, the bacterium is Gram-positive. In some embodiments, the bacterium is Gram-negative. In some embodiments, the bacterium is selected from streptococcus, haemophilus, staphylococcus and mycobacterium bacteria. In some embodiments, the bacterium is Streptococcus pneumoniae. In some embodiments, the bacterium is haemophilus influenza. In some embodiments, the bacterium is Staphylococcus aureus. In some embodiments, the bacterium is Mycobacterium tuberculosis.

In some embodiments, the virus infects mammals. In some embodiments, the virus infects humans. In some embodiments, the bacteria infects mammals. In some embodiments, the bacteria infects humans. In some embodiments, the subject is a subject susceptible to pulmonary infection. In some embodiments, the subject is a subject susceptible to respiratory infection. In some embodiments, the subject is a subject susceptible to viral infection. In some embodiments, the subject is a subject susceptible to bacterial infection.

In some embodiments, the lipotoxicity is characterized by inflammation. In some embodiments, the disease is characterized by inflammation. In some embodiments, the inflammation is inflammation of the lungs. In some embodiments, the inflammation is characterized by the overexpression of at least one cytokine or chemokine. In some embodiments, the cytokine is a pro-inflammatory cytokine. In some embodiments, the chemokine is a pro-inflammatory chemokine. In some embodiments, the cytokine or chemokine is selected from CCL20, CXCL1, CXCL2, CXCL5, GCSF, IL-1b, IL-6, IL-8, NFKB, SAA2, and TNFα. In some embodiments, GCSF is CSF3. In some embodiments, the cytokine or chemokine is CCL20. In some embodiments, the cytokine or chemokine is CXCL1. In some embodiments, the cytokine or chemokine is CXCL2. In some embodiments, the cytokine or chemokine is CXCL5. In some embodiments, the cytokine or chemokine is GCSF. In some embodiments, the cytokine or chemokine is IL-1b. In some embodiments, the cytokine or chemokine is IL-6. In some embodiments, the cytokine or chemokine is IL-8. In some embodiments, the cytokine or chemokine is NFKB. In some embodiments, the cytokine or chemokine is SAA2. In some embodiments, the cytokine or chemokine is TNF. In some embodiments, TNF is TNFa. Methods of measuring these markers of inflammation are well known in the art and are provided hereinbelow. Table 2 provides exemplary primers that can be used for measuring these markers though many other methods of measurement are known in the art and may be used. In some embodiments, expression is mRNA expression. In some embodiments, expression is protein expression. In some embodiments, expression is expression in the lung. In some embodiments, in the lung is in lung tissue. In some embodiments, the disease is a disease that can cause pneumonia. In some embodiments, the disease is a disease that can cause cytokine storm. In some embodiments, the disease is a disease that can cause acute respiratory distress syndrome (ARDS).

In some embodiments, the lipotoxicity is characterized by inhibition of a peroxisome proliferator-activated receptor (PPAR) pathway. In some embodiments, the disease is characterized by inhibition of a PPAR pathway. In some embodiments, PPAR is PPAR alpha (PPARA). In some embodiments, a PPAR pathway is a PPARA pathway. In some embodiments, inhibition of a PPAR pathway comprises reduced expression of carnitine palmitoyltransferase 1A (CPT1A). In some embodiments, inhibition of a PPAR pathway is determined by measuring expression of CPT1A. In some embodiments, reduced expression of CPT1A indicates inhibition of a PPAR pathway. In some embodiments, inhibition of a PPAR pathway comprises reduced expression of CPT1A. In some embodiments, the lipotoxicity is characterized by reduced expression of CPT1A. In some embodiments, CPT1A expression is protein expression. In some embodiments, CPT1A expression is mRNA expression. Expression can be evaluated by any method known in the art, including but not limited to, sequencing, microarray, Western blot, PCR, qPCR, protein arrays, immunostaining, histological staining/analysis and many others. In some embodiments, the reduced expressing or inhibition is in the lung of the subject. In some embodiments, the lung is lung tissue. In some embodiments, the lung is a lung sample. In some embodiments, the reduced expression or inhibition is in the subject. In some embodiments, in the subject is in a sample from the subject. In some embodiments, the sample comprises lung cells. In some embodiments, the sample is a lung biopsy. In some embodiments, the sample is from the respiratory tract. In some embodiments, the sample is acquired by noninvasive means, examples of which include lavages, washes, and swabs. In some embodiments, the sample comprises cells. In some embodiments, the cells are cells of the respiratory tract. In some embodiments, the sample is a lavage sample. In some embodiments, the lavage is bronchoalveolar lavage. In some embodiments, the lavage is whole lung lavage. In some embodiments, the sample is a nasal wash sample. In some embodiments, the sample is a swab. In some embodiments, the swab is selected from: an oral swab, a nasal swab, a tracheal swab, and a bronchial swab. In some embodiments, the sample is a blood sample. In some embodiments, the sample comprises white blood cells. In some embodiments, the white blood cells are monocytes. In some embodiments, the reduced expression or inhibition is blood. In some embodiments, the reduced expression or inhibition is in monocytes. In some embodiments, the disease or conditioned is modeled and the model shows reduced expression or inhibition. In some embodiments, the model is an animal model. In some embodiments, the model is a cellular model. In some embodiments, the cells in the model are lung cells. In some embodiments, the lung cells are isolated lung cells. In some embodiments, the lung cells are primary lung cells. In some embodiments, the lung cells are cell line cells. In some embodiments, the cells in the model are explant cells.

In some embodiments, the subject is a mammal. In some embodiments, the subject is avian. In some embodiments, the subject is feline. In some embodiments, the subject is swine. In some embodiments, the subject is a primate. In some embodiments, the subject is a human. In some embodiments, the subject is in need of treatment. In some embodiments, the subject is in need of determining suitability to be treated.

In some embodiments, the subject suffers from lung lipotoxicity. In some embodiments, the subject suffers from a disease characterized by lung lipotoxicity. In some embodiments, the subject suffers from a disease that comprises lung lipotoxicity. In some embodiments, the subject suffers from a lung infection. In some embodiments, the subject has a confirmed lung infection. In some embodiments, the subject is infected by a virus. In some embodiments, the subject has a confirmed viral infection. In some embodiments, the subject is infected by a bacterium. In some embodiments, the subject has a confirmed bacterial infection. Infection can be confirmed by any method known in the art. Commonly diagnosis of a virus is performed by a PCR test, although antigen tests can also be used. Methods for performing PCR testing are known in the art and any such method may be employed. Commonly diagnosis of a bacteria is performed by a culturing assay. In some embodiments, a PCR test is run to determine the type of bacteria.

In some embodiments, the subject was infected at most 1, 2, 3, 4, 5, 6, 7, 8, 9, or 10 days before the administering. Each possibility represents a separate embodiment of the invention. In some embodiments, the administering is immediately after diagnosis of the infection. In some embodiments, immediately is within 1, 2, 4, 5, 6, 8, 12, 18, 24, 36, 48, 72, 96, 120, 148, or 172 hours. Each possibility represents a separate embodiment of the invention. In some embodiments, immediately is within 24 hours. In some embodiments, immediately is within 48 hours.

In some embodiments, infection is symptoms onset. In some embodiments, infection is diagnosis. In some embodiments, infection is estimated to be 5 days before symptoms onset. In some embodiments, the subject was infected at most 7 days before the administering. In some embodiments, the subject was infected at most 5 days before the administering. In some embodiments, the subject was infected at most 4 days before the administering. In some embodiments, the subject was infected 3-7 days before the administering. In some embodiments, the subject was infected 3-6 days before the administering. In some embodiments, the subject was infected 3-5 days before the administering. In some embodiments, the subject was infected 3-4 days before the administering. In some embodiments, the subject was infected 4-7 days before the administering. In some embodiments, the subject was infected 4-6 days before the administering. In some embodiments, the subject was infected 4-5 days before the administering. In some embodiments, the subject was infected 5-7 days before the administering. In some embodiments, the subject was infected 5-6 days before the administering. In some embodiments, the subject was infected 6-7 days before the administering.

In some embodiments, the subject is in the first phase of an infection. In some embodiments, the first phase is the first stage. In some embodiments, the first phase is early infection. In some embodiments, the first phase is pre-symptomatic. In some embodiments, the first phase comprises upper respiratory tract infection. In some embodiments, the first phase comprises upper respiratory tract symptoms. In some embodiments, the subject is in the second phase of an infection. In some embodiments, the second phase is the second stage. In some embodiments, the second phase is the pulmonary phase. In some embodiments, the second stage comprises two parts IIA and IIB. In some embodiments, stage IIA comprises pneumonia without hypoxia. In some embodiments, stage IIB comprises pneumonia with hypoxia. In some embodiments, the second phase comprises lower respiratory tract infection. In some embodiments, the second stage comprises pneumonia. In some embodiments, the subject is in either phase 1 or phase 2.

In some embodiments, the subject is not in phase 3. In some embodiments, phase 3 is stage 3. In some embodiments, phase 3 is the hyperinflammation phase. In some embodiments, phase 3 comprises cytokine storm. In some embodiments, phase 3 comprises ARDS. In some embodiments, phase 3 comprises ICU entrance. In some embodiments, phase 3 comprises mechanical ventilation. In some embodiments, phase 3 is the acute phase of infection. In some embodiments, the subject has not yet entered an acute phase of infection.

In some embodiments, the subject is at risk of lung lipotoxicity. In some embodiments, the subject is at risk for lung disease. In some embodiments, the subject is at risk for a lung condition. In some embodiments, the subject is at risk of an infection. In some embodiments, a subject at risk is a non-vaccinated subject. In some embodiments, a subject at risk is a subject with an immunodeficiency. In some embodiments, a subject at risk is a subject with at least one comorbidity. In some embodiments, a subject at risk is a subject in a region/location with a high infection rate. In some embodiments, a high infection rate is a rate of infection above 1, 2, 3, 4, 5, 6, 7, 8, 9, 10, 11, 12, 13, 14, 15, 16, 17, 18, 19 or 20% infection. Each possibility represents a separate embodiment of the invention. In some embodiments, a high infection rate is a rate of infection above 10% infection. In some embodiments, a subject at risk is a front-line worker. In some embodiments, a subject at risk is a medical worker. In some embodiments, a subject at risk is a nursing home worker. In some embodiments, a subject at risk is a subject who cannot be vaccinated. In some embodiments, a subject at risk is anyone during a pandemic.

In some embodiments, the subject does not suffer from a metabolic disease or disorder. As used herein, the terms “metabolic disease” and “metabolic disorder” are synonymous and refer to a condition in which normal metabolism is disrupted. In some embodiments, a metabolic disease is an energy homeostasis disease. In some embodiments, the metabolic disease is a metabolic disease treatable by a metabolic regulatory drug. In some embodiments, the metabolic disease is a metabolic disease treatable by PPARA agonists. In some embodiments, the metabolic disease is a metabolic disease treatable by IRE1 pathway inhibitors. In some embodiments, the subject does not suffer from a disease treatable by a metabolic regulatory drug. In some embodiments, the subject does not suffer from a disease treatable by a PPARA agonist. In some embodiments, the subject does not suffer from a disease treatable by a IRE1 pathway inhibitor. In some embodiments, the subject does not suffer from a metabolic disease treatable by fibrates. In some embodiments, a metabolic disease treatable by a IRE1 pathway inhibitor is hypertension. In some embodiments, a metabolic disease treatable by a PPARA agonist is dyslipidemia. In some embodiments, a metabolic disease treatable by fibrates is dyslipidemia. In some embodiments, dyslipidemia comprises hyperglyceridemia. In some embodiments, dyslipidemia is hyperglyceridemia. In some embodiments, hyperglyceridemia is hypertriglyceridemia. In some embodiments, the subject is not currently being treated by a metabolic regulatory drug. In some embodiments, the subject is not currently being treated by a PPARA agonist. In some embodiments, the subject is not currently being treated by a IRE1 pathway inhibitor. In some embodiments, the subject was not previously treated by a metabolic regulatory drug. In some embodiments, the subject was not previously treated by a PPARA agonist. In some embodiments, the subject was not previously treated by a IRE1 pathway inhibitor.

In some embodiments, the metabolic disease is obesity. In some embodiments, the metabolic disease is metabolic syndrome. In some embodiments, the metabolic disease is diabetes mellitus. In some embodiments, the metabolic disease is dyslipidemia. In some embodiments, the metabolic disease is coronary heart disease. In some embodiments, the metabolic disease is hypertension. In some embodiments, the metabolic disease is hyperglyceridemia. In some embodiments, the metabolic disease is hypertriglyceridemia.

In some embodiments, the subject suffers from at least one comorbidity. In some embodiments, comorbidity is comorbidity with the lung lipotoxicity. In some embodiments, comorbidity is comorbidity with the disease. In some embodiments, the comorbidity is selected from hypertension, diabetes mellitus, coronary heart disease, cerebrovascular diseases, obesity, dyslipidemia, asthma, chronic obstructive pulmonary disease (COPD), chromic liver disease, and chronic kidney diseases. In some embodiments, the comorbidity is selected from hypertension, diabetes mellitus, coronary heart disease, cerebrovascular diseases, obesity, dyslipidemia, chromic liver disease, and chronic kidney diseases.

In some embodiments, the subject does not suffer from a comorbidity. In some embodiments, the subject does not suffer from a metabolic comorbidity. In some embodiments, the subject does not suffer from a comorbidity treatable by a metabolic regulatory drug. In some embodiments, the subject does not suffer from a comorbidity treatable by a PPAR agonist. In some embodiments, the subject is not currently being treated by a metabolic regulatory drug. In some embodiments, the subject has not previously been treated with a metabolic regulatory drug. In some embodiments, the subject is not currently being treated by a PPAR agonist. In some embodiments, the subject has not previously been treated by a PPAR agonist. In some embodiments, a subject being treated with a first metabolic regulatory drug for a condition that is not viral infection is treated with a second metabolic regulatory drug. In some embodiments, a subject already being treated by a non-PPAR agonist is treated with a PPAR agonist.

In some embodiments, a metabolic regulatory drug is administered. In some embodiments, a metabolic regulatory drug is a metabolic regulator. In some embodiments, the metabolic regulatory drug is a glycolysis inhibitor. In some embodiments, the metabolic regulatory drug is not a glycolysis inhibitor. In some embodiments, a glycolysis inhibitor inhibits at least one of GLUT1, SGLT1 and SGLT2. In some embodiments, the metabolic regulatory drug is a PPAR agonist. In some embodiments, the PPAR is PPARA. In some embodiments, the PPAR agonist is a PPARA agonist. In some embodiments, the metabolic regulatory drug is a PPARA agonist. In some embodiments, a PPAR agonist is administered. In some embodiments, a PPARA agonist is administered. In some embodiments, the PPAR is PPAR gamma (PPARG). In some embodiments, the metabolic regulatory drug is an AMPK activator. In some embodiments, the metabolic regulatory drug is not an AMPK activator. In some embodiments, the metabolic regulatory drug is an alpha-glucosidase inhibitor. In some embodiments, the metabolic regulatory drug is an HNGCR inhibitor. In some embodiments, the metabolic regulatory drug is not an HNGCR inhibitor. In some embodiments, the metabolic regulatory drug is a statin. In some embodiments, the metabolic regulatory drug is not a statin. In some embodiments, the metabolic regulatory drug is a C/EBP inhibitor. In some embodiments, the metabolic regulatory drug is an ER stress inhibitor. In some embodiments, the metabolic regulatory drug is a IRE1 pathway antagonist. In some embodiments, the metabolic regulatory drug is a thiazolidinedione. In some embodiments, the metabolic regulatory drug is not a thiazolidinedione.

In some embodiments, a composition is administered. In some embodiments, the composition is a pharmaceutical composition. In some embodiments, the composition is a therapeutic composition. In some embodiments, the composition comprises a metabolic regulatory drug. In some embodiments, the composition comprises the PPAR agonist. In some embodiments, the composition comprises the PPARA agonist. In some embodiments, the composition comprises a pharmaceutically acceptable carrier, excipient or adjuvant. In some embodiments, the composition is formulated for oral administration. In some embodiments, the composition is formulated for systemic administration. In some embodiments, the composition is formulated for intravenous administration.

In some embodiments, the metabolic regulatory drug is a PPARA agonist. In some embodiments, the PPARA agonist is a PPARA specific agonist. In some embodiments, the PPARA agonist does not agonize PPAR gamma (PPARG). In some embodiments, the PPARA agonist does not significantly agonize PPARG. In some embodiments, the PPARA agonist produce a greater agonizing effect on PPARA than on PPARG. In some embodiments, the greater effect is at least a 2-fold, 3-fold, 4-fold, 5-fold, 6-fold, 7-fold, 8-fold, 9-fold, or 10-fold greater effect. Each possibility represents a separate embodiment of the invention. In some embodiments, the greater effect is at least a 10-fold greater effect. In some embodiments, the PPARA agonist does agonize PPARG. In some embodiments, a PPARA agonist is a small molecule agonist. In some embodiments, a PPARA agonist is a PPARA activator. In some embodiments, the PPARA agonist is a fibrate. Fibrates are well known in the art and any known fibrate may be used. Examples of fibrates include, but are not limited to fenofibrate, bezafibrate, gemfibrozil, pemafibrate, and ciprofibrate. In some embodiments, the PPARA agonist is pirinixic acid. In some embodiments, pirinixic acid is WY-14,643. In some embodiments, the PPARA agonist is a conjugated linoleic acid (CLA). In some embodiments, the PPARA agonist is MD001. In some embodiments, the PPARA agonist is LY518674. In some embodiments, the PPARA agonist is K111. In some embodiments, the PPARA agonist is ZYH7. In some embodiments, the PPARA agonist is selected from a fibrate, pirinixic acid and a CLA. In some embodiments, the PPARA agonist is selected from a fibrate, pirinixic acid, a CLA, MD001, LY518674, K111, ZYH7, and Macuneos.

CLAs are well known in the art and include at least 28 isomers of linoleic acid. In some embodiments, the CLA is a mix of isomers. In some embodiments, the isomer is 9-CLA. In some embodiments, 9-CLA is rumenic acid. In some embodiments, the isomer is 10-CLA. In some embodiments, the CLA is 9-CLA. In some embodiments, the CLA is 10-CLA. In some embodiments, the CLA is selected from 9-CLA and 10-CLA.

Fibrates are a class of amphipathic carboxylic acids that are well known in the art. In some embodiments, a fibrate treats a metabolic disorder. In some embodiments, a fibrate treats high cholesterol (hypercholesterolemia). In some embodiments, a fibrate treats hyperglyceridemia. In some embodiments, a fibrate treats dyslipidemia. In some embodiments, a fibrate treats hypertriglyceridemia. In some embodiments, treating is the drug's primary treatment. In some embodiments, the fibrate is selected from aluminum clofibrate, bezafibrate, ciprofibrate, choline fenofibrate, clinofibrate, clofibrate, clofibride, fenofibrate, gemfibrozil, ronifibrate and simfibrate. In some embodiments, the fibrate is selected from aluminum clofibrate, bezafibrate, ciprofibrate, choline fenofibrate, clinofibrate, clofibrate, clofibride, fenofibrate, gemfibrozil, ronifibrate, fenofibric acid, pemafibrate and simfibrate. In some embodiments, the fibrate is selected from Fenofibrate (Fenoglide/Tricor), Gemfibrozil (Lopid), Clofibrate (Atromid-S), Clinofibrate (Lipoclin), Bezafibrate (Bezalip), Simfibrate, Ronifibrate, Clofibride, fenofibric acid, pemafibrate, and Ciprofibrate. In some embodiments, the fibrate is fenofibrate. In some embodiments, the fibrate is not gemfibrozil. In some embodiments, the fibrate is selected from fenofibrate, bezafibrate, gemfibrozil, pemafibrate, and ciprofibrate.

In some embodiments, the metabolic regulatory drug is a PPARG agonist. In some embodiments, the PPARG agonist is a PPARG specific agonist. In some embodiments, the PPARG agonist does not agonize PPARA. In some embodiments, the PPARG agonist does not significantly agonize PPARA. In some embodiments, the PPARG agonist produces a greater agonizing effect on PPARG than on PPARA. In some embodiments, the greater effect is at least a 2-fold, 3-fold, 4-fold, 5-fold, 6-fold, 7-fold, 8-fold, 9-fold, or 10-fold greater effect. Each possibility represents a separate embodiment of the invention. In some embodiments, the greater effect is at least a 10-fold greater effect. In some embodiments, the PPARG agonist does agonize PPARA. In some embodiments, the PPARA agonist is selected from Genistein (Isoflavone), Luteolin (Lavone) and Imatinib (Gleevec). In some embodiments, the PPARG inhibitor is a C/EBP inhibitor. In some embodiments, the C/EBP inhibitor is selected from Genistein (Isoflavone), Luteolin (Lavone) and Imatinib (Gleevec). In some embodiments, the PPARG agonist is a PPARG activator. In some embodiments, the PPARG agonist is selected from the TZD group of PPARG activators. In some embodiments, the TZD group comprises Pioglitazone (Actos), Rosiglitazone (Avandia) and Lobeglitazone (Duvie). In some embodiments, the metabolic regulatory drug is not a PPARG agonist.

In some embodiments, the metabolic regulatory drug is a IRE1 pathway inhibitor. In some embodiments, the IRE1 pathway inhibitor is an inhibitor of IRE1 alpha (IRE1a). In some embodiments, an IRE1 pathway inhibitor inhibits ER stress. In some embodiments, a IRE1 pathway inhibitor treats hypertension. In some embodiments, the IRE1 pathway inhibitor is telmisartan. In some embodiments, the IRE1 pathway inhibitor is selected from telmisartan, Sunitinib, STF-083010, 4u8C, KIRA6m, Kira8, Kira7, MKC8866, GSK2850163, Toyocamycin, APY29, MKC3946, MKC9989, NSC95682, B-I09, 3,6-DMAD, and IRE1α kinase-IN-2. In some embodiments, the metabolic regulatory drug is not a IRE1 pathway inhibitor.

In some embodiments, the metabolic regulatory drug is an ER stress inhibitor. ER stress inhibitors are well known in the art and include for example Quercetin (flavonoid), Salubrinal, TUDCA (bile acid), and UDCA (Actigall). In some embodiments, the metabolic regulatory drug is not an ER stress inhibitor.

In some embodiments, the metabolic regulatory drug is a statin. Statins are well known in the art and include for example simvastatin (Zocor) and Pravastatin (Pravachol) among many others. In some embodiments, a statin is a HMG-COA reductase inhibitor. In some embodiments, a statin is a high cholesterol treatment. In some embodiments, a statin is a diabetes mellitus treatment. In some embodiments, the metabolic regulatory drug is not a statin.

In some embodiments, the metabolic regulatory drug is a glycolysis inhibitor. In some embodiments, the glycolysis inhibitor is an inhibitor of at least one of GLUT1, SGLT2 and SGLT1. In some embodiments, the glycolysis inhibitor is an inhibitor of SGLT2. In some embodiments, a glycolysis inhibitor is an inhibitor of glucose transport. Such inhibitors are well known in the art and include, but are not limited to Dapagliflozin (Forxiga), Canagliflozin (Invokana), Empagliflozin (Jardiance), Ertugliflozin (Steglatro), Sotagliflozin (Zynquista), Quinidine, Cloperastine (Hustazol), Bepridil, Trihexyphenidyl, Bupivacaine, (+)-ε-viniferin, (+)-pteryxin, BAY 876, WZB-117, STF-31, and Fasentin. In some embodiments, the metabolic regulatory drug is not a glycolysis inhibitor.

In some embodiments, the metabolic regulatory drug is an AMPK activator. In some embodiments, the AMPK activator is metformin. AMPK activators are well known in the art and include for example metformin, phenformin (DBI), buformin (Silubin), Proguanil, Chlorproguanil and AICAR. In some embodiments, the metabolic regulatory drug is not an AMPK activator.

In some embodiments, the metabolic regulatory drug is an alpha-glucosidase inhibitor. Alpha-glucosidase inhibitors are well known in the art and include for example Acarbose, Miglitol and Voglibose. In some embodiments, the regulatory drug is not an alpha-glucosidase inhibitor.

In some embodiments, a metabolic regulatory drug is not a thiazolidinedione. In some embodiments, a metabolic regulatory drug is not metformin. In some embodiments, a metabolic regulatory drug is not an AMPK activator. In some embodiments, a metabolic regulatory drug is not a statin. In some embodiments, a metabolic regulatory drug is not a glycolysis inhibitor.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

In some embodiments, treating comprises treating at least one symptom of lung lipotoxicity. In some embodiments, treating comprises treating at least one symptom of the disease or disorder. Symptoms of respiratory diseases are well known in the art, and include, but are not limited to, fever, cough, runny nose, fatigue, muscle aches, sore throat, headache, difficulty breathing, shortness of breath, chest pain, chest pressure, loss of speech, and disorientation. Ir some embodiments, treating comprises reducing phospholipid accumulation. In some embodiments, the accumulation in within a lung of the subject. In some embodiments, the accumulation in within lung tissue. In some embodiments, the accumulation is within lung cells. In some embodiments, the lung cells are lung epithelial cells. In some embodiments, lung cells are bronchiolar cells. In some embodiments, lung cells are tracheal cells. In some embodiments, lung cells are alveolar cells. In some embodiments, treating comprises reducing viral load. In some embodiments, viral load is viral load in the subject. In some embodiments, treating comprises reducing symptoms. In some embodiments, treating comprises reducing inflammation. In some embodiments, inflammation is inflammation in the subject. In some embodiments, inflammation is systemic inflammation. In some embodiments, inflammation is lung inflammation. In some embodiments, inflammation is characterized by levels of C-reactive protein (CRP). In some embodiments, treating comprises reducing CRP levels. In some embodiments, treating comprises reducing the risk of entering phase 3 of the disease. In some embodiments, treating comprises reducing a risk of mechanical ventilation. In some embodiments, mechanical ventilation is invasive mechanical ventilation. In some embodiments, treating comprises reducing a risk of septic shock. In some embodiments, treating comprises reducing a risk of acute liver injury. In some embodiments, treating comprises reducing a risk of acute kidney injury. In some embodiments, treating comprises reducing a risk of acute cardiac injury. In some embodiments, treating comprises reducing a risk of ICU admission. In some embodiments, treating comprises reducing a risk of hospitalization. In some embodiments, treating comprises reducing hospitalization time. In some embodiments, treating comprises reducing hospitalization length. In some embodiments, treating comprises reducing a risk of developing Acute respiratory distress syndrome (ARDS). In some embodiments, treating comprises reducing a risk of developing a cytokine storm. In some embodiments, treating comprises reducing risk of death. In some embodiments, treating comprises reducing death.

In some embodiments, treating occurs within 10, 9, 8, 7, 6, 5, 4, 3, 2 or 1 day. Each possibility represents a separate embodiment of the invention. In some embodiments, treating occurs within 5 days. In some embodiments, treating occurs in 3-5 days. In some embodiments, treating occurs in 1-7, 1-6, 1-5, 1-4, 1-3, 1-2, 2-7, 2-6, 2-5, 2-4, 2-3, 3-7, 3-6, 3-5, 3-4, 4-7, 4-6, or 4-5 days. Each possibility represents a separate embodiment of the invention. In some embodiments, treating occurring is treating starting. In some embodiments, treating occurring is treating completing. In some embodiments, at least one symptom improves within the above recited time. In some embodiments, the subject is discharged from the hospital within the above recited time. In some embodiments, the subject is cured with the above recited time.

In some embodiments, decreasing the risk of symptomatic infection is causing asymptomatic infection. In some embodiments, decreasing the risk of symptomatic infection results in asymptomatic infection. In some embodiments, decreasing the risk of symptomatic infection is preventing symptomatic infection. In some embodiments, preventing symptomatic infection is in a subject that is not currently infected. In some embodiments, preventing symptomatic infection is a result of prophylactic treatment.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. In some embodiments, the administration is systemic administration. In some embodiments, the administration is oral administration. In some embodiments, the administration is intravenous administration. Other suitable routes of administration can include parenteral, subcutaneous, intravenous, aerosol, or intraperitoneal.

The dosage administered will be dependent upon the age, health, and weight of the recipient, kind of concurrent treatment, if any, frequency of treatment, and the nature of the effect desired. In some embodiments, the dose of the metabolic regulatory drug is the same dose at which it is administered to treat a metabolic condition or disease. In some embodiments, the dose administered to treat a metabolic condition or disease is the standard dose. In some embodiments, the dose of the metabolic regulatory drug is a higher dose than the dose it is administered to treat a metabolic condition or disease. In some embodiments, the dose of the metabolic regulatory drug is the same dose as for its primary treatment. In some embodiments, the dose of the metabolic regulatory drug is higher than for its primary treatment. In some embodiments, a primary treatment is a metabolic disease or condition. In some embodiments, a higher dose is a more frequent dose. In some embodiments, a PPARA agonists primary treatment is hypercholesterolemia. In some embodiments, a PPARA agonists primary treatment is hyperglyceridemia. In some embodiments, a PPARA agonists primary treatment is dyslipidemia. In some embodiments, a PPARA agonists primary treatment is hypertriglyceridemia.

In some embodiments, a higher dose is twice the dose. In some embodiments, twice the does is twice as frequently. In some embodiments, the twice as frequently is twice a day. In some embodiments, twice the dose is a single administration of a dose that is twice the standard dose. In some embodiments, a higher dose is administered on the first day of treatment and the standard dose is administered on subsequent days. In some embodiments, twice the dose is administered on the first day of treatment and the standard dose is administered on subsequence days. It will be understood by a skilled artisan, that in order to reach Cmax as quickly as possible and increased dose can be administered at first and then the standard dose can be used to maintain Cmax in the subject. In some embodiments, the dose is a dose selected from one provided in Table 1. In some embodiments, the standard dose is a dose selected from one provided in Table 1. In some embodiments, the dose for a primary treatment is a dose selected from one provided in Table 1. In some embodiments, the dose of fenofibrate is 40 to 120 mg/day. In some embodiments, the dose of fenofibrate is 40 to 150 mg/day. In some embodiments, the dose of fenofibrate is about 46 mg/day. In some embodiments, the dose of fenofibrate is about 145 mg/day. In some embodiments, the fenofibrate is nano-crystallized Fenofibrate. In some embodiments, the dose of nano-crystallized Fenofibrate is about 145 mg/day. In some embodiments, 145 mg/day is oral 145 mg/day. In some embodiments, the fenofibrate is fenofibric acid. In some embodiments, the dose of fenofibric acid is 135 mg/day. In some embodiments, 135 mg/day is oral 135 mg/day. In some embodiments the dose of fenfibric acid is 50 mg/day. In some embodiments, 50 mg/day is IV 50 mg/day. In some embodiments, the duration of the dosing is for 10 days. In some embodiments, IV dosing is for 10 days.

TABLE 1
Standard dosing for metabolic drugs
(mg/kg/day is the dose in rodents)

| | |
|---|---|
| Drug | Dose |

Glycolosis Inhibitors

| Dapagliflozin (Forxiga) | 5 to 10 | mg/day |
|---|---|---|
| Canagliflozin (Invokana) | 100 to 300 | mg/day |
| Empagliflozin (Jardiance) | 10 to 25 | mg/day |
| Ertugliflozin (Steglatro) | 5 to 15 | mg/day |
| Sotagliflozin (Zynquista) | 200 to 400 | mg/day |
| Quinidine | 200 to 800 | mg/day |
| Cloperastine (Hustazol) | 8 to 60 | mg/day |
| Bepridil | 200 to 400 | mg/day |
| Trihexyphenidyl | 1 to 15 | mg/day |
| Bupivacaine | 0.25% to 0.9% solution | |
| (+)-ε-viniferin | 48 | μM |
| (+)-pteryxin | 12 | μM |
| BAY 876 | 1.5-4.5 | mg/kg/day |
| WZB-117 | 10-30 | mg/kg/day |
| STF-31 | 4-30 | mg/kg/day |
| Fasentin | 5-60 | mg/kg/day |

PPARA Agonists

| Fenofibrate nanonised (Tricor-3) | 50 to 145 | mg/day |
|---|---|---|
| Fenofibrate micronized (Tricor-2) | 60 to 160 | mg/day |
| Fenofibrate (Tricor-1) | 60 to 200 | mg/day |
| Fenofibric acid (Trilipix) | 45 to 135 | mg/day |
| Gemfibrozil (Lopid) | 600 mg/12 hrs | |
| Clofibrate (Atromid-S) | 500 mg/6 hrs | |
| Clinofibrate (Lipoclin) | 400 to 800 | mg/day |
| Bezafibrate (Bezalip) | 400 | mg/day |
| 9CLA | 1000 | mg/day |

AMPK Activators

| Metformin | 500 mg twice a day or | |
|---|---|---|
| | 850 mg once a day | |
| Phenformin (DBI) | 100 to 800 | mg/day |
| Buformin (Silubin) | 150 to 800 | mg/day |
| Proguanil | 400 to 1000 | mg/day |
| Chlorproguanil | 15 to 240 | mg/day |
| AICAR | 20-200 | mg/kg/day |

Statins

| | | |
|---|---|---|
| Pravastatin (Pravachol) | 10 to 80 | mg/day |
| Simvastatin (Zocor) | 5 to 80 | mg/day |

In some embodiments, the metabolic regulatory drug is formulated to reach a Cmax in said subject within 1 day from administration. In some embodiments, the metabolic regulatory drug is formulated to reach a Cmax in said subject within 2 days from administration. In some embodiments, the metabolic regulatory drug is formulated to reach a Cmax in said subject within 3 days from administration. In some embodiments, metabolic regulatory drug is formulated to reach a Cmax in said subject rapidly. In some embodiments, the metabolic regulatory drug is administered in order to reach a Cmax in the subject rapidly. In some embodiments, rapidly is within 12 hours, 18 hours, 24 hours, 2 days, 3 days, 4 days or 5 days. Each possibility represents a separate embodiment of the invention. In some embodiments, rapidly is within 1 day. In some embodiments, rapidly is within 3 days. In some embodiments, rapidly is within 5 days. In some embodiments, rapidly is before onset of severe disease. In some embodiments, severe disease is acute disease. In some embodiments, acute disease is severe disease. In some embodiments, severe disease comprises cytokine storm. In some embodiments, severe disease comprises ARDS. In some embodiments, severe disease comprises mechanical ventilation.

In some embodiments, the dosing is transient dosing. In some embodiments, the treating is transient treatment. In some embodiments, transient treating comprises administering the metabolic regulatory drug for a limited number of days. In some embodiments, the dosing is for a limited number of days. In some embodiments, a limited number of days is at most 7, 8, 9, 10, 11, 12, 13, 14, 15, 16, 17, 18, 19, 20, 21, 28, 35, 42, 49, 56, 63, 70, 77, 84, 90 or 91 days. Each possibility represents a separate embodiment of the invention. In some embodiments, a limited number of days is about 7, 8, 9, 10, 11, 12, 13, 14, 15, 16, 17, 18, 19, 20, 21, 28, 35, 42, 49, 56, 63, 70, 77, 84, 90 or 91 days. Each possibility represents a separate embodiment of the invention.

It will be understood by a skilled artisan that the therapeutic agent needs to take effect before the subject reaches a point at which the therapy is no longer effective. However, the window between diagnosis and severe disease is often very short, as such, Cmax must be reached rapidly. Certain formulations are known to result in higher bioavailability and thus in a Cmax reached more rapidly. For example, it is known that many fibrates take a long time to reach Cmax, however, certain fibrate formulations (e.g., nanocrystal formulations) are known to reach Cmax more rapidly (e.g., within a day). In some embodiments, the formulation is a nanocrystal formulation. In some embodiments, the fibrate is a fibrate nanocrystal. In some embodiments, the fibrate is a fenofibrate nanocrystal. Fenofibrate nanocrystals are known in the art and include for example Tricor® and Triglide®. In some embodiments, the fenofibrate is selected from Tricor® and Triglide®. Intravenous administration is the most rapid way to reach Cmax. In some embodiments, the administration is intravenous administration. In some embodiments, the formulation is an intravenous formulation. Fibrates are generally administered orally, however, intravenous formulations are known in the art. Oral formulations of fibrates are well known in the art and are provided, for example in Ling et al., “A review of currently available fenofibrate and fenofibric acid formulations” 2013, Cardiol. Res.; 4 (2): 47-55, herein incorporated by reference in its entirety. An intravenous fenofibric acid formulation is disclosed for example in Zhu et al., “Comparison of the gastrointestinal absorption and bioavailability of fenofibrate and fenofibric acid in humans”, 2010, Journal of Clinical Pharmacology, 50:914-921, herein incorporated by reference in its entirety.

In some embodiments, the method comprises measuring lipotoxicity in a lung of the subject. In some embodiments, the method comprises confirming lipotoxicity in a lung of the subject. In some embodiments, the method comprises determining if lipotoxicity is present in a lung of the subject. In some embodiments, the presence of lipotoxicity indicates the subject is suitable to be treated by a metabolic regulatory drug. In some embodiments, the presence of lipotoxicity indicates the subject is suitable to be treated by a method of the invention. In some embodiments, the measuring is before the administering. In some embodiments, the confirming is before the administering. In some embodiments, the determining is before the administering. In some embodiments, the method comprises selecting a subject with lung lipotoxicity. In some embodiments, the method comprises a patient selection step. In some embodiments, the method comprises administering the metabolic drug to a subject confirmed to have lung lipotoxicity.

Methods of detecting, measuring or confirming lipotoxicity are well known in the art and can include any known test or assay. In some embodiments, detecting lipotoxicity comprises detecting lipids in the lungs. In some embodiments, detecting lipotoxicity comprises detecting lipids in lung tissue. In some embodiments, detecting lipotoxicity comprises detecting lipids in a sample. In some embodiments, the sample is from the respiratory tract. In some embodiments, the sample is from the lungs. In some embodiments, the sample is sputum. In some embodiments, the sample is a blood sample. In some embodiments, the sample is a spit sample. In some embodiments, the detecting is staining for lipids. Lipid specific dyes are well known in the art and include for example Oil-red-O (ORO).

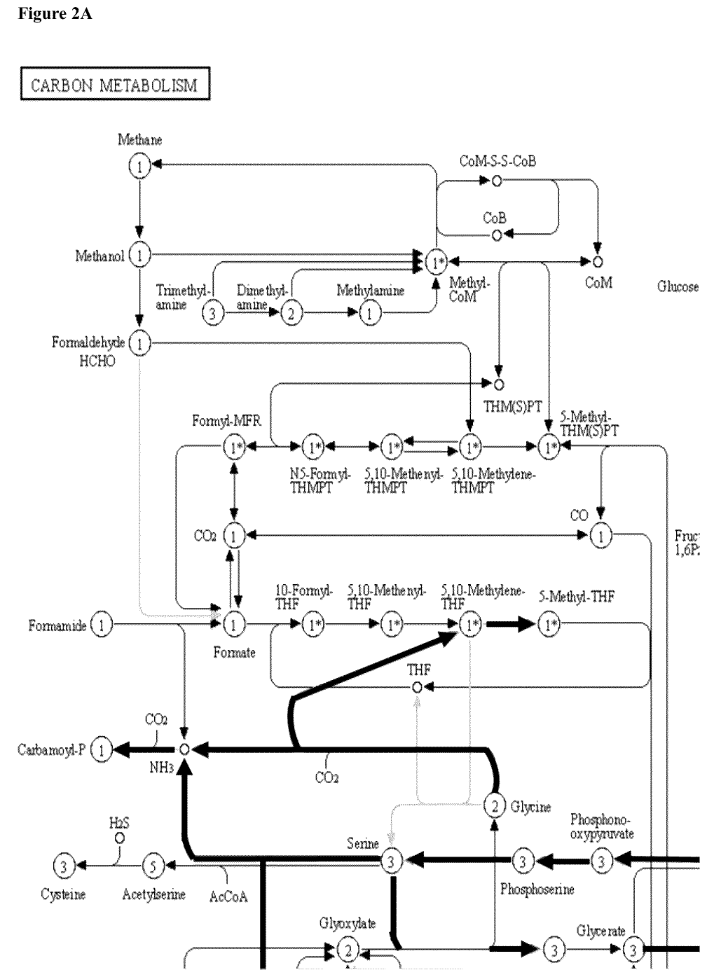

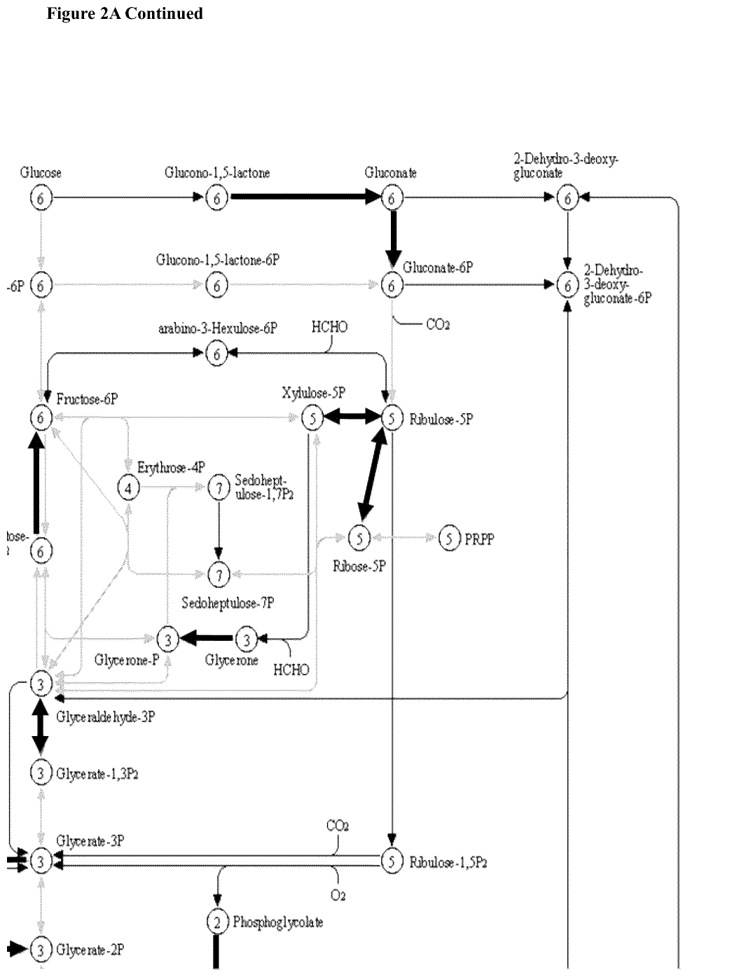

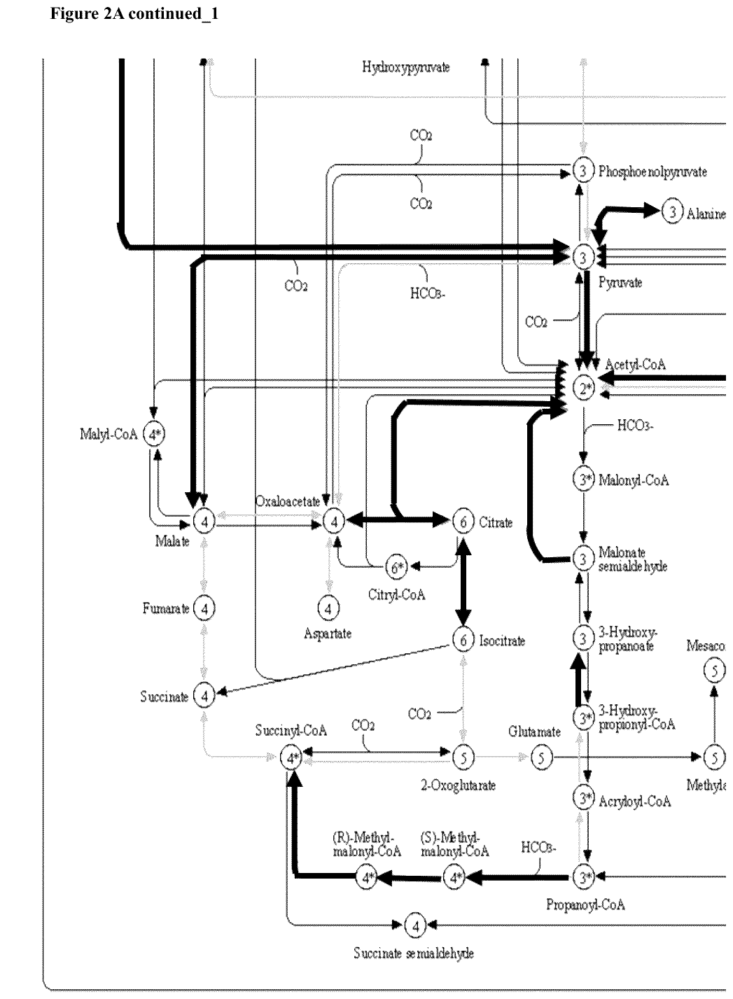

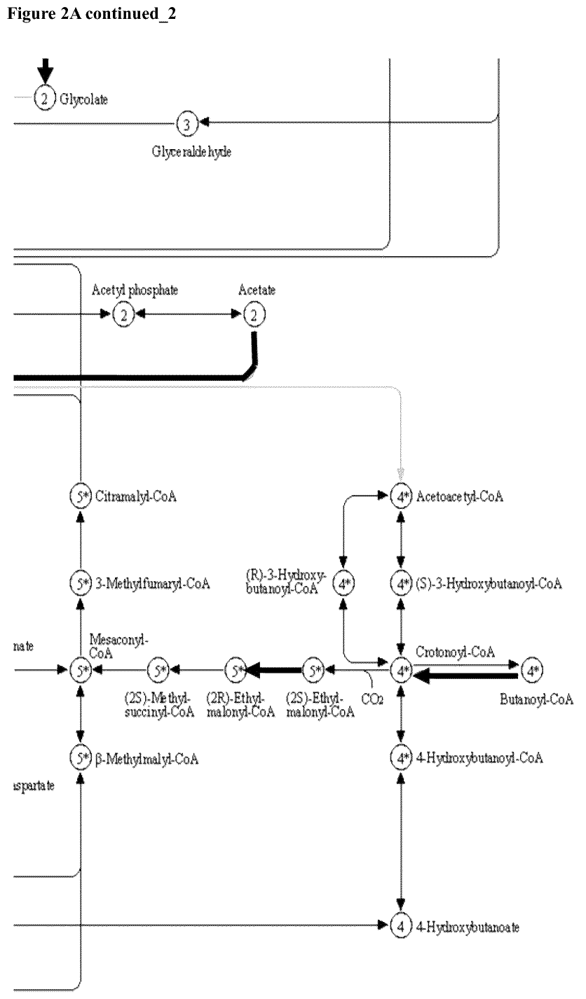

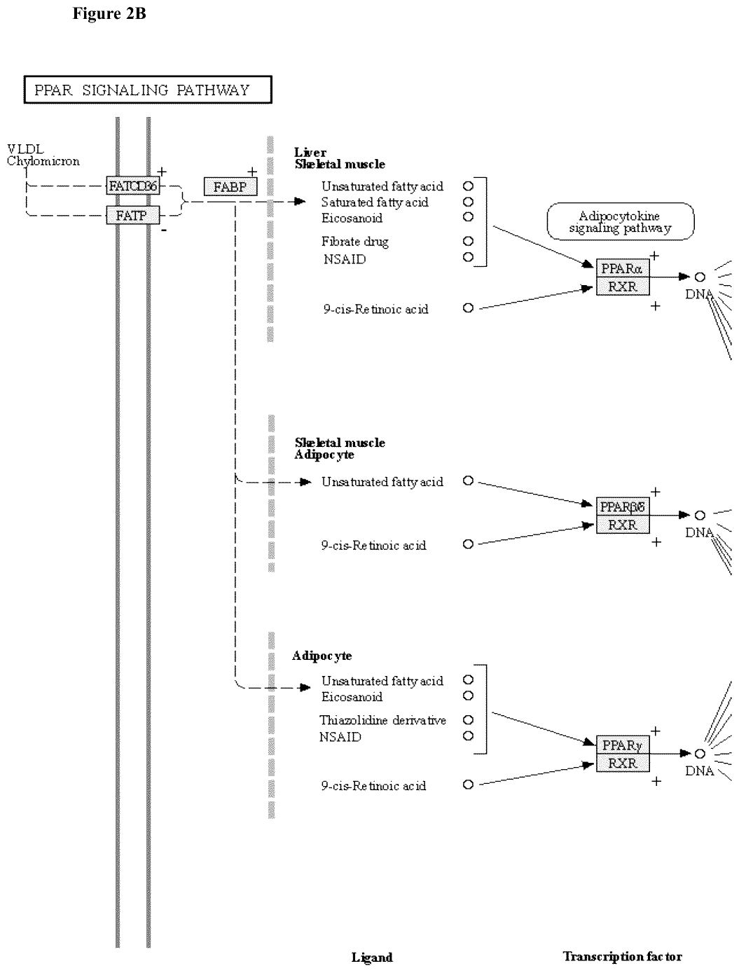

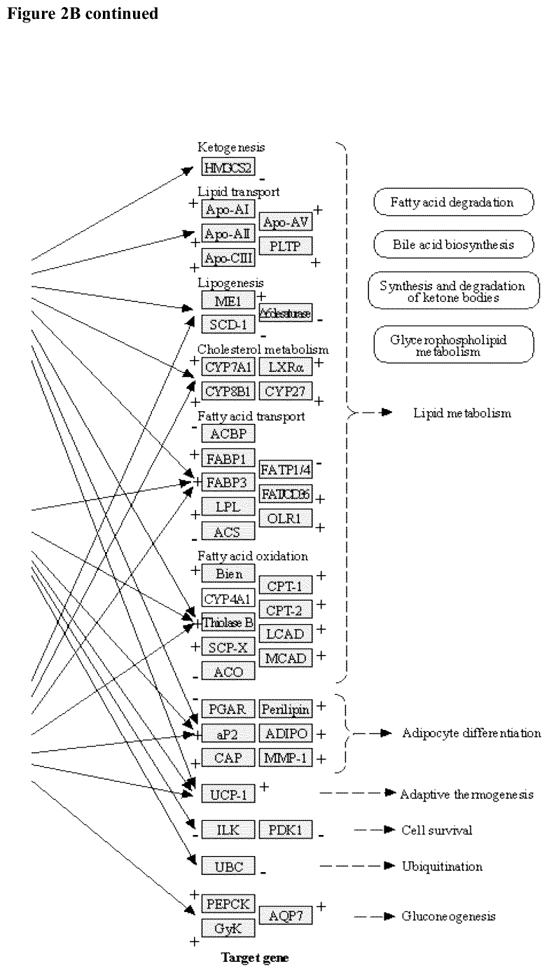

In some embodiments, detecting, measuring or confirming comprises detecting, measuring or confirming inhibition of a PPAR pathway. In some embodiments, the pathway is a PPARA pathway. In some embodiments, detecting, measuring or confirming comprises detecting, measuring or confirming reduced expression of a gene in a PPAR pathway. In some embodiments, reduced expression is downregulatory. In some embodiments, reduced is as compared to a healthy subject or sample. In some embodiments, reduced is as compared to a subject or sample from a subject that does not have the disease. In some embodiments, is reduced as compared to a subject or sample from a subject that does not have lipotoxicity of the lungs. In some embodiments, reduced is significantly reduced. In some embodiments, reduced is reduced to below a predetermined threshold. In some embodiments, the predetermined threshold is the expression in a healthy sample or subject. In some embodiments, the predetermined threshold is the expression in a subject or sample from a subject that does not have lipotoxicity of the lungs. In some embodiments, the predetermined threshold is the expression in a subject or sample from a subject that does not have the disease. In some embodiments, decreased is decreased by at least a predetermined amount. In some embodiments, the decrease is at least a 5, 10, 15, 20, 25, 30, 35, 40, 45, 50, 55, 60, 65, 70, 75, 80, 85, 90, 95, 97, 99 or 100% reduction in expression. Each possibility represents a separate embodiment of the invention. In some embodiments, the gene of the PPAR pathway is CPT1A. In some embodiments, confirming comprises confirming reduced expression of CPT1A. In some embodiments, the gene of the pathway is a gene provided in FIG. 2A or FIG. 2B. In some embodiments, the expression is protein expression. In some embodiments, the expression is mRNA expression. In some embodiments, detecting, measuring or confirming comprises detecting, measuring or confirming an increase in ER stress. In some embodiments, the confirming is in the subject. In some embodiments, the confirming is in the lung. In some embodiments, the confirming is in lung tissue. In some embodiments, the confirming is in lung cells.

In some embodiments, the confirming comprises a non-invasive method of confirming lung lipotoxicity. In some embodiments, the non-invasive method comprises acquiring a sample. In some embodiments, the confirming is performed in the sample. In some embodiments, the sample comprises cells. In some embodiments, the sample is acquired by noninvasive means, examples of which include lavages, washes, and swabs. In some embodiments, the sample comprises cells. In some embodiments, the cells are cells of the respiratory tract. In some embodiments, the sample is a lavage sample. In some embodiments, the lavage is bronchoalveolar lavage. In some embodiments, the lavage is whole lung lavage. In some embodiments, the sample is a nasal wash sample. In some embodiments, the sample is a swab. In some embodiments, the swab is selected from: an oral swab, a nasal swab, a tracheal swab, and a bronchial swab. In some embodiments, the sample is a blood sample. In some embodiments, the sample comprises white blood cells. In some embodiments, the white blood cells are monocytes. In some embodiments, the reduced expression or inhibition is blood. In some embodiments, the confirming is in monocytes.

By another aspect, there is provided a method of determining if a subject is suitable to be treated by a method of the invention, the method comprising measuring lipotoxicity in a lung of a subject, wherein the presence of lipotoxicity in the lung indicates the subject is suitable to be treated by a method of the invention.

By another aspect, there is provided a method of determining if a subject is suitable to be treated by a metabolic regulatory drug, the method comprising measuring lipotoxicity in a lung of a subject, wherein the presence of lipotoxicity in the lung indicates the subject is suitable to be treated by a metabolic regulatory drug.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Cell Culture: NHBE cells (ATCC, PCS-300-010 Lot #63979089; #70002486), isolated from a 69-year-old Caucasian male and a 14-year-old Hispanic male were maintained in airway epithelial cell basal media (ATCC, PCS-300-030) supplemented with Bronchial Epithelial Growth Kit as per the manufacturer's instructions (ATCC, PCS-300-040) at 37° C. and 5% CO2.

Analysis of gene expression by RNAseq: Expression count matrices were retrieved from GEO. Differential gene expression analysis was performed using a Poisson-Tweedie distribution model using the tweeDEseq Bioconductor package. Count data from GEO were normalized using a trimmed-mean of M values (TMM) normalization with the edgeR Bioconductor packages. Genes with the following criteria were considered differentially expressed: (1) P-value adjusted by Benjamini-Hochberg (B&H) method FDR<0.05, (2) A fold change>1.25, (3) Minimal mean expression>20 in either condition.

Analysis of Canonical Splice Variants: Reads were downloaded from SRA (GSE147507) and filtered and trimmed to remove low-quality reads and sequencing artifacts with fastp v20 (github.com/OpenGene/fastp.git). Reads were pseudoaligned to the GRCh38 genecode human transcriptome (GRCh38.p13, version 32) using Kallisto version 0.46.1 (github.com/pachterlab/kallisto) run with the default k-mer length of 31, in single-read, single-overhang mode, with fragment mean length of 400 and 100 SD. Differentially expressed transcripts/genes were identified using Sleuth based on a likelihood ratio test comparing the condition of interest and 100 Kallisto bootstrap samples.

Quantification of Lipids: Lipid accumulation was measured using HCS LipidTOX Phospholipidosis and Steatosis Detection Kit according to the manufacturer's instructions (ThermoFisher, USA; H34158). Briefly, cells were incubated in complete bronchial epithelial growth media supplemented with 1× phospholipidosis detection reagent for 48 hours. Cells were subsequently fixed in 4% PFA and stained with 1× neutral lipid detected reagent for 30 min and counterstained with 1 μg mLHoechst 33258. Staining intensity was normalized to the amount of Hoechst 33258 positive nuclei across multiple fields of view.

Generation PPARα CRISPR knock-out cells: The PPARα knock-out cells were created using a Cas9-based, CRISPR system. Two different sgRNA oligos from the human GeCKO v.2 Human CRISPR Knockout Pooled Library (Addgene; #1000000048), PPARα HGLibA 37838 and HGLibB_37787, were cloned into the lentiCRISPR v2 plasmid (Addgene; #52961). The sgRNA cloning was performed according to the human GeCKO v.2 system instructions. Briefly, two oligos comprising each sgRNA insert were synthesized with BsmBI-compatible ends, and the vector plasmid was digested with BsmBI (FastDigest Esp3I, FD0454, Thermo), de-phosphorylated (FastAP thermosensitive alkaline phosphatase, EF0651, Thermo), and gel extracted (QiaQuick gel extraction, Qiagen). The sgRNA oligos were phosphorylated and annealed in a single session: first phosphorylation using T4 PNK (NEB-M0201S) followed by heating to 95° C. for 5 min and controlled cooling to allow annealing. The vector and insert fragments were ligated (T4 DNA ligase, EL0011) and transformed into chemical competent Stb13 cells (Mix & Go! E. coli Transformation Kit, T3001, Zymo). Correctly ligated plasmids were used for lentiviral sgRNA vector production. Approximately 1×10\hat{ }6 cells were infected in two consecutive sessions of 12 h each. The cells were then selected using 3 μM puromycin for 72 hours (Merck; P9620).

Assessment of Immunoinflammatory Markers by Quantitative Real-time PCR Analysis: Total RNA was extracted and isolated using a NucleoSpin RNA II kit (Macherey-Nagel, Germany) according to the manufacturer's instructions. RNA concentration and purity were determined using NanoDrop ND-1000 spectrophotometer (Thermo Fisher Scientific, USA). cDNA synthesis was performed using qScript™ cDNA Synthesis (Quanta BioSciences) according to the manufacturer's instructions. 1 μg of purified RNA was used for each reaction, with concentration and purity determined by an ND-1000 spectrophotometer (NanoDrop Technologies). Each reaction was diluted to reach a concentration of 10 ng/μL. mRNA expression levels were measured by qRT-PCR using KAPA SYBR FAST (Kapa Biosystems) on Applied Biosystem QuantStudio 5 Real-Time PCR System. Gene transcription was evaluated using the AACt method normalized to ribosomal protein L32 (RPL32) and ubiquitin-conjugating enzyme (UBC1). Primer sequences and sources are listed in Table 2.

TABLE 2
Primer sequences

| | | |
|---|---|---|
| Gene | Forward Primer (SEQ ID NO:) | Reverse Primer (SEQ ID NO:) |

Housekeeping genes

| | | |
|---|---|---|
| b-Actin | 5′-ATCATGTTTGAGACCTTCAAC-3′ (1) | 5′-CATCTCTTGCTCGAAGTCCA-3′ (2) |
| RPL32 | 5′-ACAAAGCACATGCTGCCCAGTG-3′ (3) | 5′-TTCCACGATGGCTTTGCGGTTC-3′ (4) |
| UBL2 | 5′-CGGGTGTGGCACAGCTAGTT-3′ (5) | 5′-TGCATTGTCAAGTGACGATCAC-3′ (6) |

Immunoinflammatory Stress

| | | |
|---|---|---|
| CCL20 | 5′-GGACATAGCCCAAGAACAGAAA-3′ (7) | 5′-GTCCAGTGAGGCACAAATTAGA-3′ (8) |
| CXCL1 | 5′-TGTTCTAAATATCCCTTGGACA-3′ (9) | 5′-CCTTTGTTCTAAGCCAGAAAC-3′ (10) |
| CXCL2 | 5′-TATTGGTGGCTGTTCCTGAAG-3′ (11) | 5′-CTCAAACACATTAGGCGCAATC-3′ (12) |
| CXCL5 | 5′-GTAGAACCTGGGTTAGAGGATTG-3′ (13) | 5′-CCAGTGAAATGAACTGTGCTAAA-3′ (14) |
| GCSF | 5′-TGCCTGGACATTTGCCTT-3′ (15) | 5′-ACAGTGGAGCTTCCTTTCAC-3′ (16) |
| IL-1b | 5′-CTCTCACCTCTCCTACTCACTT-3′ (17) | 5′-TCAGAATGTGGGAGCGAATG-3′ (18) |
| NFKB2 | 5′-GTGAAGACCTTGCTGCTAAATG-3′ (19) | 5′-GGTTCTGCAGAGCTGTATCA-3′ (20) |
| SAA2 | 5′-TCCTCTTCACTCTGCTCTCA-3′ (21) | 5′-GCCATATCTCAGCTTCTCTGG-3′ (22) |
| IL-6 | 5′-CCCTGACCCAACCACAAA-3′ (23) | 5′-GGACTGCAGGAACTCCTTAAA-3′ (24) |
| IL-8 | 5′-AAATCTGGCAACCCTAGTCTG-3′ (25) | 5′-GTGAGGTAAGATGGTGGCTAAT-3′ (26) |
| TNFa | 5′-AGCTTTGATCCCTGACATCTG-3′ (27) | 5′-TCTAGGTGAGGTCTTCTCAAGT-3′ (28) |

Functional Annotations of Gene Expression: Differentially expressed genes were tested for enrichment overlap within functional gene sets. The general test for functional enrichment of the differentially expressed genes against various functional categories was done using the PANTHER tool. Enrichment P values were calculated using Fisher's exact test and corrected with familywise (Bonferroni) multiple testing correction or the Benjamini-Hochberg False discovery method as indicated.

Western blot: NHBE, PPARα CRISPR-KO NHBE cells or PPARa-OE HEK293T cells were washed in DPBS, lysed in 1×Laemmli Loading buffer and boiled at 100° C.; 40 μl of cleared lysate were analysed in a pre-cast gradient polyacrylamide gel (Bolt 4 to 12%, Bis-Tris, 1.0 mm, Mini Protein Gel/NW04120BOX, Invitrogen) using SeeBlue Plus2 Pre-stained Protein Standard (LC5925, Invitrogen) in MES SDS running buffer (B0002, Invitrogen) according to manufacturer's instructions. The proteins were transferred to a PVDF membrane (iBlot 2 Transfer Stacks, PVDF, mini/IB24002, Invitrogen) using iBlot2 (LifeSciences). The membrane was blocked with 5% BSA (160069, MPBio) in Tris-buffered saline plus 0.1% Tween 20 (TBST) for 1 h at room temperature. The membranes were incubated in primary antibodies overnight at 4° C. The next day, the membranes were washed in TBST (3×10 min) and then incubated with horseradish peroxidase-conjugated secondary antibody for 2 h at room temperature. After the TBST washes (4×10 min), EZ-ECL kit (Sartorius; 20-500-1000A, 20-500-1000B) was used to detect the HRP activity. The membrane was imaged on a Vilber Fusion FX and band densitometry was performed on FIJI.

The following commercial primary antibodies were used: anti-PPARα (1:1000; ab24509, Abcam) and anti-α-tubulin (1:2000; T6074, Sigma). Commercial horseradish peroxidase—conjugated secondary antibodies were: anti-rabbit (111-035-003, Jackson) and anti-mouse (115-035-003, Jackson). All primary antibodies were used in 5% BSA in TBST. Secondary antibodies were used at a 1:8,000 dilution in TBST.

### Example 1: The Metabolic Fingerprint of Influenza

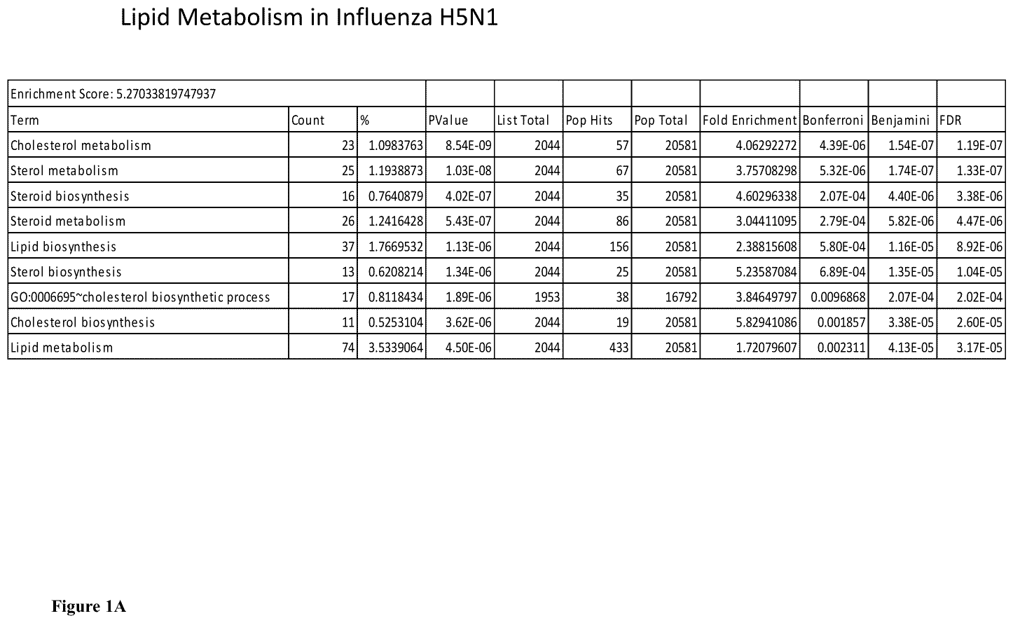

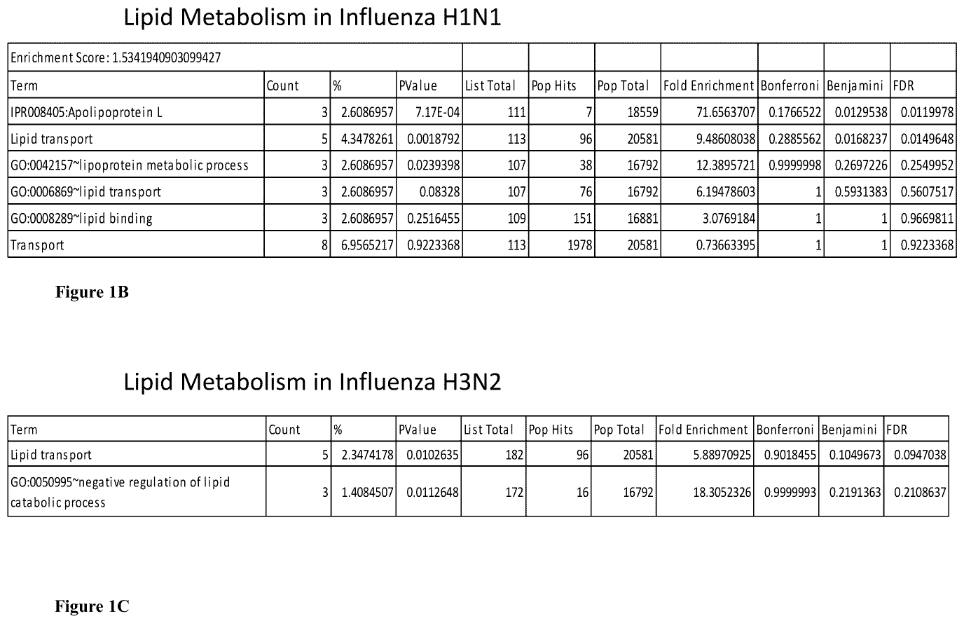

To elucidate the metabolic effects of influenza, human tracheobronchial epithelial (HTBE; LONZA, DONOR 4F1289J) cells were infected with three strains of influenza virus (H5N1 (bird flu), HIN1, and H3N1 (swine flu)). After 18 hours RNA was isolated from infected and uninfected cells and RNA-Seq analysis was performed. Differentially expressed genes were identified for each virus strain (FDR<0.05), and enrichment analysis was run using DAVID functional annotation software. All three strains showed significant differences in lipid metabolism just 18 hours post infection (FIG. 1A-C). Interestingly, all the strains showed changes in metabolic processes, particularly lipid metabolic processes.

Next, the differentially expressed genes of the H5N1 strain of influenza were mapped on the central carbon metabolism (FIG. 2A) and PPAR signaling (FIG. 2B) pathways using KEGG Mapper algorithm (KEGG Pathway database). Gene mapping shows significant changes in the expression of genes relating to glucose metabolism, lipid metabolism and mitochondrial respiration. Mapping genes on the PPAR signaling pathway showed changes in lipid oxidation and synthesis.

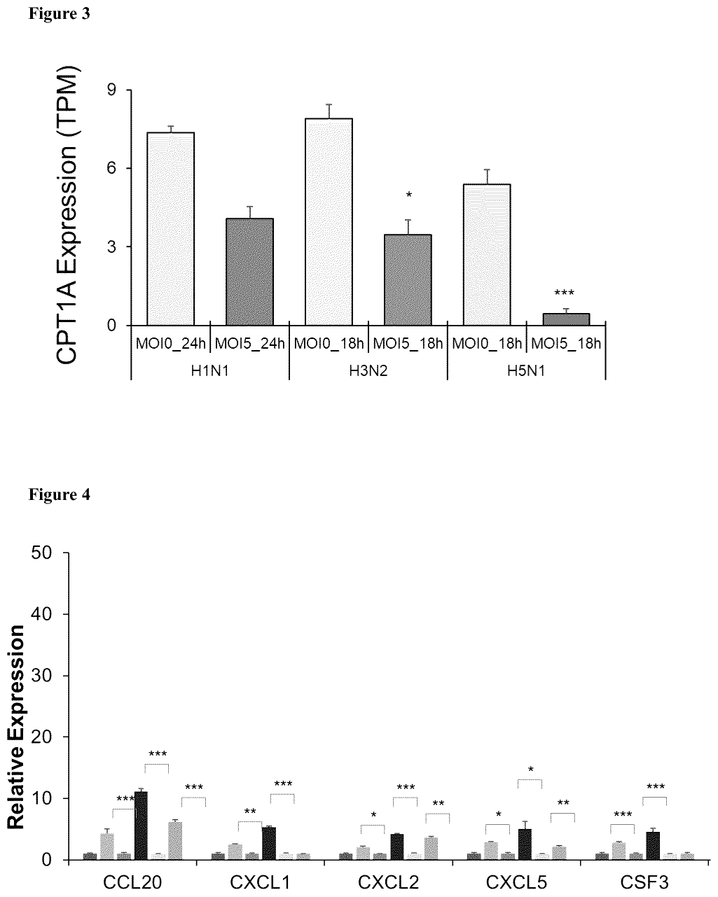

In order to test whether PPAR mediated lipid oxidation is a hallmark of influenza in general, HTBEcells were infected with the three strains of influenza at either a multiplicity of infection (MOI) of zero or an MOI of 5. The zero infection is a mock infection that acts as control cells, whereas the MOI of 5 resulted in greater than 99% of cells being infected. After 24 hours (H1N1) or 18 hours (H3N2 and H5N1) the cells were sequenced by RNA-Seq, aligned, quantified by Kallisto and analyzed by Sleuth. A significant inhibition in transcription of CPT1A, a key rate limiting enzyme in PPARA mediated fatty acid oxidation, was induced by all three strains (FIG. 3).

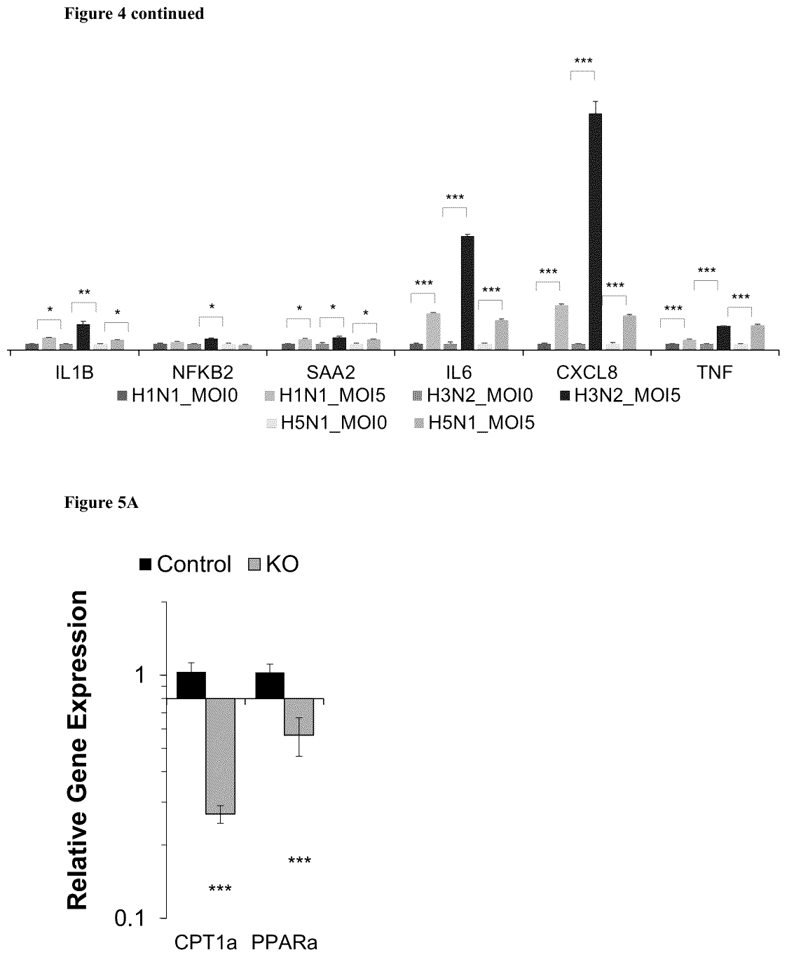

Next, HTBE cells were infected with the three influenza strains and expression of markers of immunoinflammatory stress response were measured by qRT-PCR. Infection was performed at MOI-0 (no infection), MOI-5. Significant upregulation of CCL20, CXCL2, CXCL5, IL-1B, SAA2, IL-6, CXCL8 and TNF was observed upon infection with all 3 influenzas (FIG. 4). Significant upregulation of CXCL1, and CSF3 was observed only upon infection with H1N1 and H3N2 and significant upregulation of NFKB2 was observed only upon infection with H3N2.

### Example 2: PPARα KO Leads to Lipid Accumulation and CPT1α Reduction

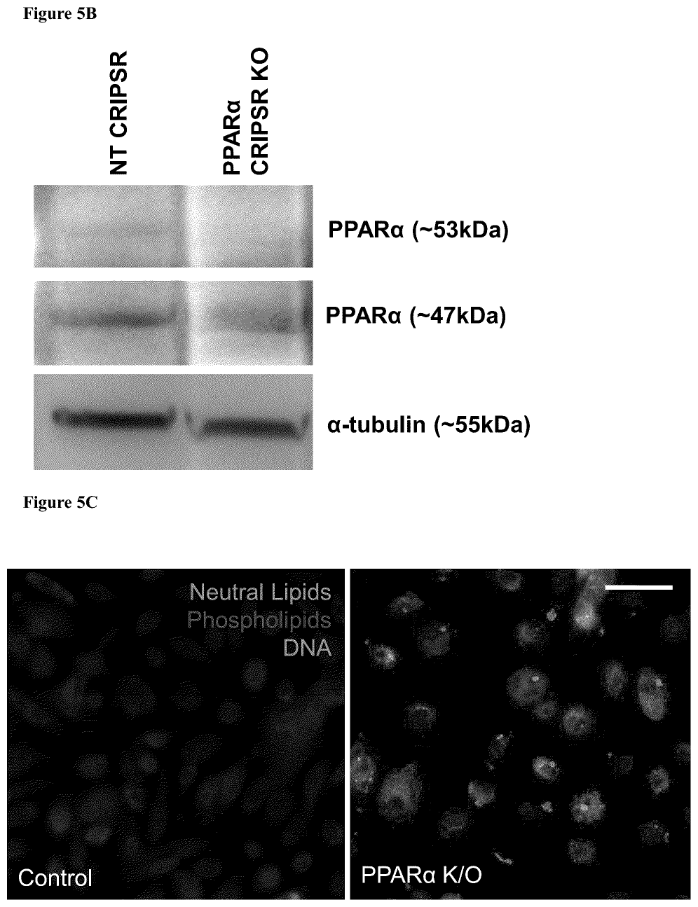

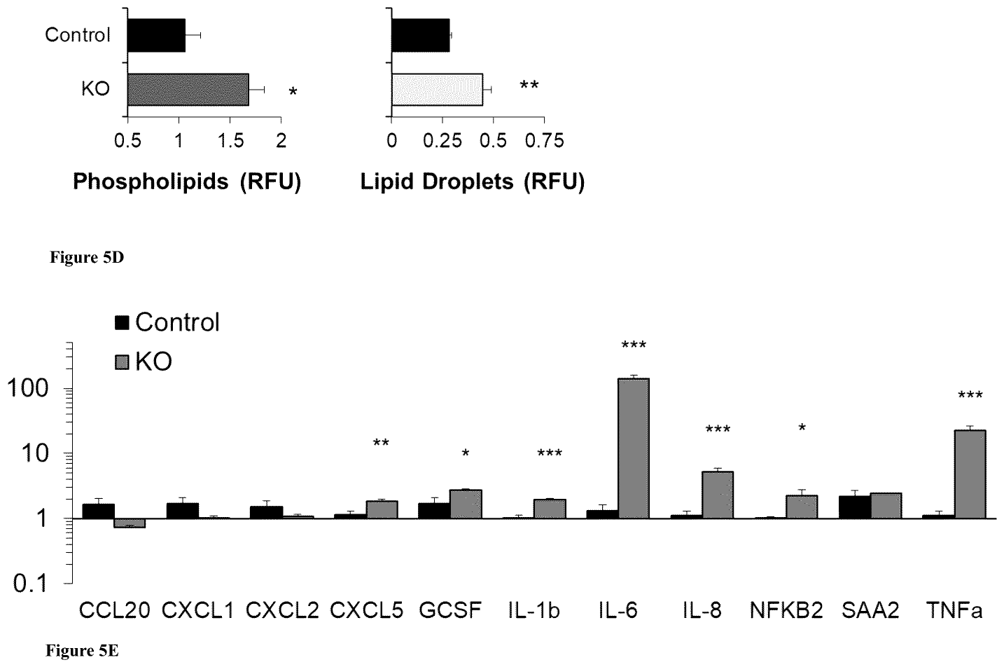

Based on the lipid signature observed in influenza infected lung cells, PPARα protein was CRISPR knocked out (KO) in the bronchial epithelial cells. mRNA (FIG. 5A) and protein knockdown (FIG. 5B) were confirmed by qPCR and western blot, respectively, after 5 days of selection for the CRISPR construct. KO of PPARα resulted in lipid accumulation in the bronchial cells (FIG. 5C) which represented a 50% increase in triglycerides (n=6, p<0.05) and a 60% increase in phospholipids (n=6, p<0.001) (FIG. 5D) as compared to the WT cells. KO of PPARα also resulted in a pronounced downregulation of lipid oxidation regulator CPT1a (FIG. 5A). Further, a similar upregulation of markers of immunoinflammatory stress response was also observed in the KO lung cells (FIG. 5E). It is thus apparent that loss of PPARα expression/function mimics the deleterious lipotoxic effects of influenza infection.

### Example 3: PPARγ Agonism Leads to Lipid Accumulation and CPT1α Reduction

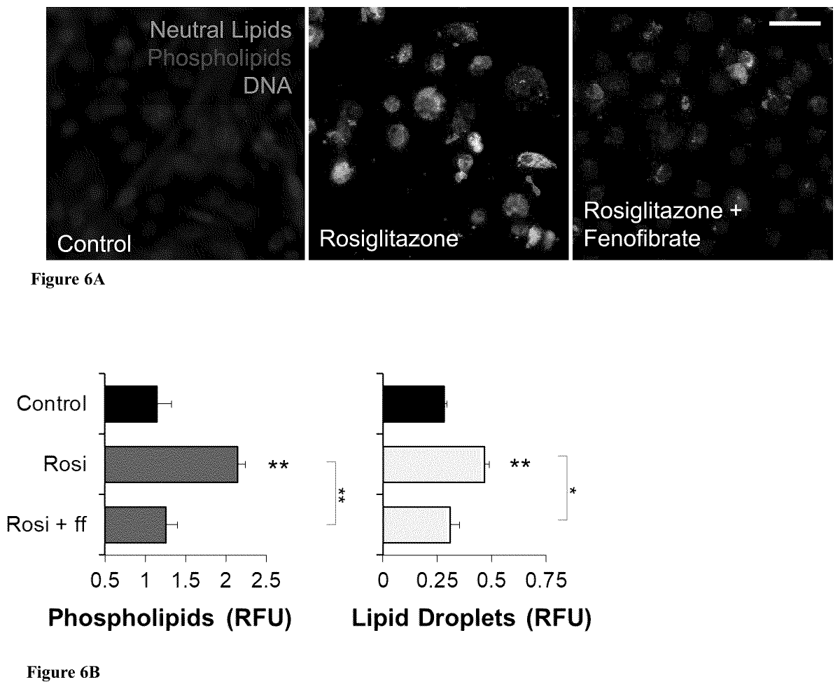

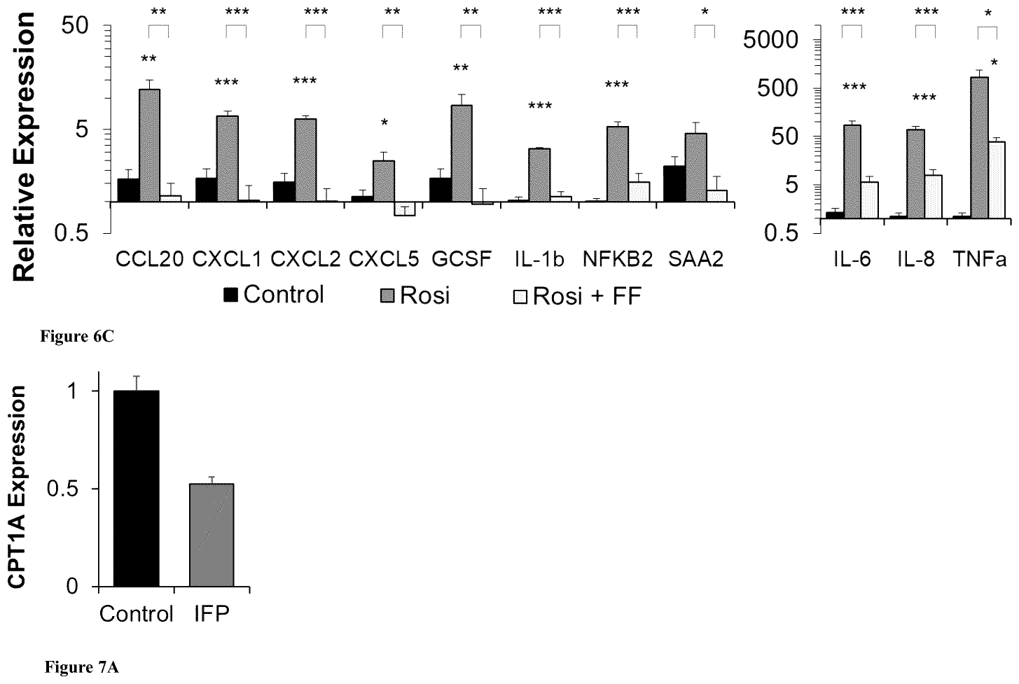

Next, instead of knocking out PPARα agonism of PPARγ was examined. Bronchial epithelial cells were induced by 10 μM of PPARγ agonist rosiglitazone and 100 μM oleic acid for 5 days. As expected, lipid accumulation was observed in the lung cells (FIG. 6A) with a 65% increase in triglycerides (n=6, p<0.05) and 75% increase in phospholipids (n=6, p<0.001) as compared to untreated cells (FIG. 6B). Rosiglitazone also produced the same upregulation of markers of immunoinflammatory stress response (FIG. 6C). Importantly, the addition of the PPRAα agonist fenofibrate was able to not only completely reverse the lipid accumulation (FIG. 6A-B) but also abrogated the toxicity/immunoinflammation that it produced (FIG. 6C). Indeed, fenofibrate addition led to lower levels of several inflammatory markers (CCL20, CXCL1, CXCL2, CXCL5, GCSF, and SAA2) then even were observed in the control cells. This demonstrates the effectiveness of fenofibrate as a treatment for lipotoxicity in lung cells.

### Example 4: CPT1α Reduction and the Lipotoxicity Inflammatory Signal is Present in Other Lung Diseases

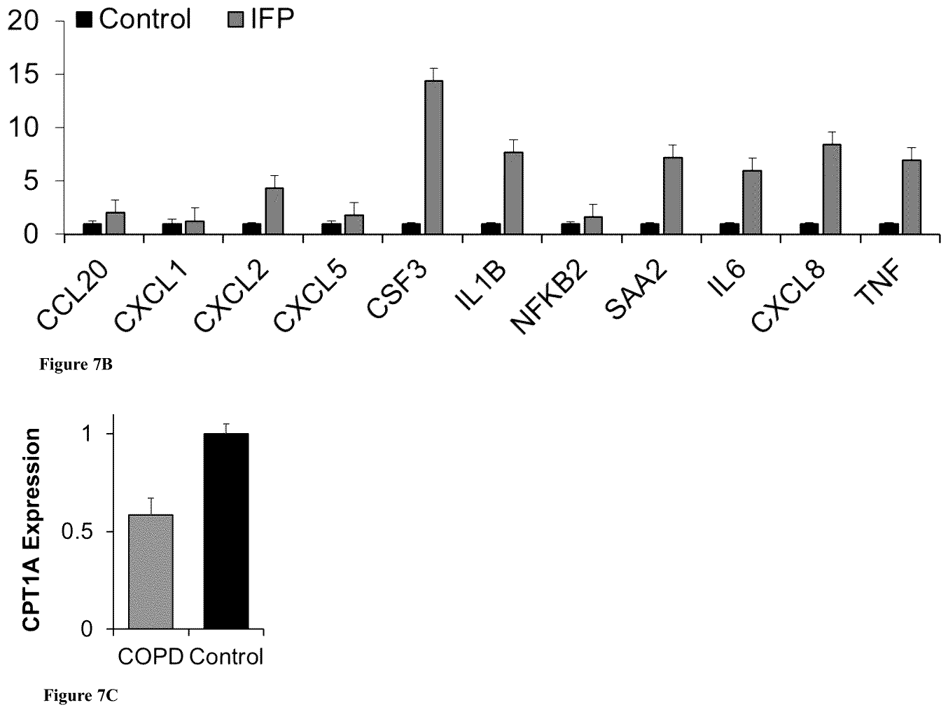

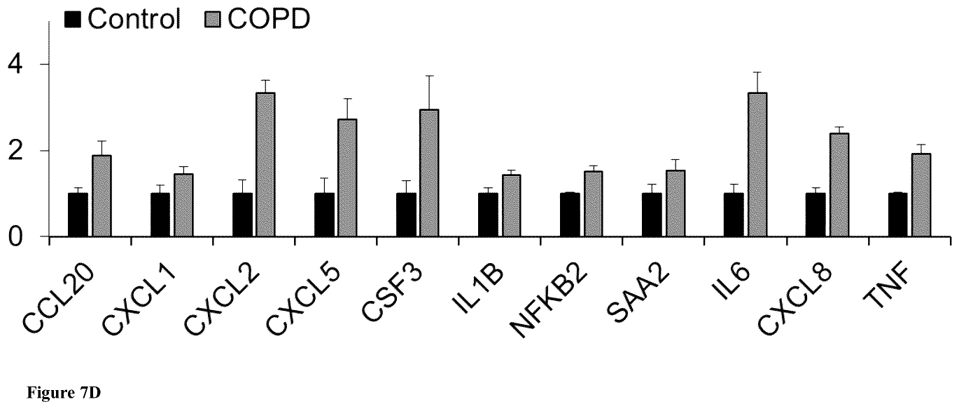

As reduced CPT1A levels are indicative of lipid accumulation, other lung conditions were examined for this same hallmark of lipotoxicity. The Gene Expression Omnibus (GEO) database entry GSM5207232 contains mRNA expression data from qRT-PCR of fixed paraffin-embedded (FFPE) lung tissue from healthy non-fibrotic lung tissue and lung tissue from subjects with Idiopathic Pulmonary Fibrosis (IPF). The data from 10 patients of each type was averaged. CPT1A levels were indeed significantly reduced in the IFP subjects (FIG. 7A) indicating that at least some of the subjects were suffering from lung lipotoxicity. This was supported by the presence of upregulation of CXCL2, CSF3, IL1B, SAA2, IL-6, CXCL8 and TNF (FIG. 7B). It is possible that other markers of immunoinflammation may have been upregulated in some subject but not in others and thus did not appear significantly upregulated.

GEO database entry GSE162154 contains mRNA expression data from qRT-PCR from isolated human small airway epithelial cells (HSAEC) isolated from lung tissue of subjects that never smoked and subjects suffering from Chronic obstructive pulmonary disease (COPD). Once again CPT1A levels were significantly reduced in the COPD subjects (FIG. 7C) and an upregulation of markers of immunoinflammation was also observed (FIG. 7D). Similar markers of lung lipotoxicity have also been reported for or can be found in published gene expression data for infant respiratory distress syndrome (IRDS), acute respiratory distress syndrome (ARDS/RDS), acute lung injury (ALI), vaping-associated lung injury, pulmonary alveolar proteinosis (PAP), pneumonia and tuberculosis (TB). This demonstrates that other lung diseases are characterized by lung lipotoxicity and that certain lung disease patients suffer from lung lipotoxicity as part of their pathology. Such diseases and patients can therefore be treated with metabolic regulatory drugs.

To validate the effect of metabolic regulators on lung lipotoxicity in a clinical setting, patients with confirmed influenza infection and admitted to hospital are broken down based on their being treated at the time with a metabolic regulatory drug. Subjects being treated with PPARA agonists have better outcomes. It should be noted that one patient with pulmonary hypertension did not respond to treatment with fibrates. Though this subject showed high CRP levels indicating inflammation, pulmonary hypertension is not associated with lipotoxicity in the lungs.

Effects of metabolic regulators, in particular PPARA agonists, and specifically fenofibrate are also confirmed in an animal model of lung lipotoxicity. Administration of the regulator produces better outcomes, reduces inflammation and increases CPT1A levels.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating lipotoxicity in a lung of a subject in need thereof, the method comprising administering to said subject a therapeutic composition comprising at least one metabolic regulatory drug, thereby treating lipotoxicity in the lungs in a subject.

2. (canceled)

3. The method of claim 1, wherein said lipotoxicity is at least one of:
a. characterized by a reduction in carnitine palmitoyltransferase 1A (CPT1A) in lung tissue from said subject;
b. caused by a bacterial or viral infection of said lung;
c. caused by an influenza infection of said lung;
d. caused by an influenza H1N1, H3N2 or H5N1 infection of said lung.

4. (canceled)

5. (canceled)

6. The method of claim 3, wherein said lipotoxicity is caused by bird or swine influenza infection of said lung.

7. (canceled)

8. The method of claim 3, wherein said bacterial or viral infection produces pneumonia in said lung, is not a coronavirus infection or both.

9. (canceled)

10. The method of claim 1, wherein said disease is selected from influenza, idiopathic pulmonary fibrosis (IPF), chronic obstructive pulmonary disease (COPD), infant respiratory distress syndrome (IRDS), acute respiratory distress syndrome (ARDS/RDS), acute lung injury (ALI), vaping-associated lung injury, pulmonary alveolar proteinosis (PAP), pneumonia and tuberculosis (TB).

11. The method of claim 10, wherein said disease is selected from influenza, IPF and COPD.

12. The method of claim 1, further comprising confirming lipotoxicity in said lung of said subject before administering said therapeutic composition.

13. The method of claim 12, wherein said confirming comprises confirming inhibition of a peroxisome proliferator-activated receptor (PPAR) pathway or reduced expression of a gene in said PPAR pathway or confirming reduced expression of CPT1A.

14. The method of claim 13, wherein said PPAR is PPAR alpha (PPARA).

15. The method of claim 14, wherein said confirming comprises confirming reduced expression of CPT1A.

16. (canceled)

17. The method of claim 1, wherein said subject is not currently or was not previously treated with said metabolic regulatory drug, does not suffer from a metabolic disease or disorder treatable by said regulatory drug.

18. (canceled)

19. The method of claim 1, wherein said metabolic regulatory drug is a PPAR agonist.

20. The method of claim 19, wherein said PPAR agonist is a PPARA agonist.

21. The method of claim 20, wherein said PPARA agonist is selected from a fibrate, pirinixic acid and conjugated linoleic acid (CLA) and derivatives thereof.

22. The method of claim 21, wherein said CLA is selected from 9-CLA and 10-CLA.

23. The method of claim 21, wherein said fibrate is selected from aluminum clofibrate, bezafibrate, ciprofibrate, choline fenofibrate, clinofibrate, clofibrate, clofibride, fenofibrate, gemfibrozil, ronifibrate, fenofibric acid, pemafibrate, and simfibrate.

24. The method of claim 23, wherein said fibrate is fenofibrate or is not gemfibrozil.

25. (canceled)

26. The method of claim 1, wherein said administering is oral or intravenous administering.

27. The method of claim 1, wherein said metabolic regulatory drug is administered on the first day of administration at twice a dose administered for treating a metabolic condition and is subsequently administered at said dose for treating a metabolic condition.

28. The method of claim 1, wherein said treating comprises at least one of reduced phospholipid accumulation in lung cells, reduced viral load, reduced symptoms, reduced inflammation, reduced risk of Acute respiratory distress syndrome (ARDS), reduced risk of a cytokine storm and reduced risk of death.

29. (canceled)


SEQUENCE LISTING (28 sequences)

SEQ ID NO: 1 - DNA, 21 bp, synthetic construct
  misc_feature 1..21: note = Synthetic
  source 1..21: mol_type = other DNA
atcatgtttgagaccttcaac

SEQ ID NO: 2 - DNA, 20 bp, synthetic construct
  misc_feature 1..20: note = Synthetic
  source 1..20: mol_type = other DNA
catctcttgctcgaagtcca

SEQ ID NO: 3 - DNA, 22 bp, synthetic construct
  misc_feature 1..22: note = Synthetic
  source 1..22: mol_type = other DNA
acaaagcacatgctgcccagtg

SEQ ID NO: 4 - DNA, 22 bp, synthetic construct
  misc_feature 1..22: note = Synthetic
  source 1..22: mol_type = other DNA
ttccacgatggctttgcggttc

SEQ ID NO: 5 - DNA, 20 bp, synthetic construct
  misc_feature 1..20: note = Synthetic
  source 1..20: mol_type = other DNA
cgggtgtggcacagctagtt

SEQ ID NO: 6 - DNA, 22 bp, synthetic construct
  misc_feature 1..22: note = Synthetic
  source 1..22: mol_type = other DNA
tgcattgtcaagtgacgatcac

SEQ ID NO: 7 - DNA, 22 bp, synthetic construct
  misc_feature 1..22: note = Synthetic
  source 1..22: mol_type = other DNA
ggacatagcccaagaacagaaa

SEQ ID NO: 8 - DNA, 22 bp, synthetic construct
  misc_feature 1..22: note = Synthetic
  source 1..22: mol_type = other DNA
gtccagtgaggcacaaattaga

SEQ ID NO: 9 - DNA, 22 bp, synthetic construct
  misc_feature 1..22: note = Synthetic
  source 1..22: mol_type = other DNA
tgttctaaatatcccttggaca

SEQ ID NO: 10 - DNA, 21 bp, synthetic construct
  misc_feature 1..21: note = Synthetic
  source 1..21: mol_type = other DNA
cctttgttctaagccagaaac

SEQ ID NO: 11 - DNA, 21 bp, synthetic construct
  misc_feature 1..21: note = Synthetic
  source 1..21: mol_type = other DNA
tattggtggctgttcctgaag

SEQ ID NO: 12 - DNA, 22 bp, synthetic construct
  misc_feature 1..22: note = Synthetic
  source 1..22: mol_type = other DNA
ctcaaacacattaggcgcaatc

SEQ ID NO: 13 - DNA, 23 bp, synthetic construct
  misc_feature 1..23: note = Synthetic
  source 1..23: mol_type = other DNA
gtagaacctgggttagaggattg

SEQ ID NO: 14 - DNA, 23 bp, synthetic construct
  misc_feature 1..23: note = Synthetic
  source 1..23: mol_type = other DNA
ccagtgaaatgaactgtgctaaa

SEQ ID NO: 15 - DNA, 18 bp, synthetic construct
  misc_feature 1..18: note = Synthetic
  source 1..18: mol_type = other DNA
tgcctggacatttgcctt

SEQ ID NO: 16 - DNA, 20 bp, synthetic construct
  misc_feature 1..20: note = Synthetic
  source 1..20: mol_type = other DNA
acagtggagcttcctttcac

SEQ ID NO: 17 - DNA, 22 bp, synthetic construct
  misc_feature 1..22: note = Synthetic
  source 1..22: mol_type = other DNA
ctctcacctctcctactcactt

SEQ ID NO: 18 - DNA, 20 bp, synthetic construct
  misc_feature 1..20: note = Synthetic
  source 1..20: mol_type = other DNA
tcagaatgtgggagcgaatg

SEQ ID NO: 19 - DNA, 22 bp, synthetic construct
  misc_feature 1..22: note = Synthetic
  source 1..22: mol_type = other DNA
gtgaagaccttgctgctaaatg

SEQ ID NO: 20 - DNA, 20 bp, synthetic construct
  misc_feature 1..20: note = Synthetic
  source 1..20: mol_type = other DNA
ggttctgcagagctgtatca

SEQ ID NO: 21 - DNA, 20 bp, synthetic construct
  misc_feature 1..20: note = Synthetic
  source 1..20: mol_type = other DNA
tcctcttcactctgctctca

SEQ ID NO: 22 - DNA, 21 bp, synthetic construct
  misc_feature 1..21: note = Synthetic
  source 1..21: mol_type = other DNA
gccatatctcagcttctctgg

SEQ ID NO: 23 - DNA, 18 bp, synthetic construct
  misc_feature 1..18: note = Synthetic
  source 1..18: mol_type = other DNA
ccctgacccaaccacaaa

SEQ ID NO: 24 - DNA, 21 bp, synthetic construct
  misc_feature 1..21: note = Synthetic
  source 1..21: mol_type = other DNA
ggactgcaggaactccttaaa

SEQ ID NO: 25 - DNA, 21 bp, synthetic construct
  misc_feature 1..21: note = Synthetic
  source 1..21: mol_type = other DNA
aaatctggcaaccctagtctg

SEQ ID NO: 26 - DNA, 22 bp, synthetic construct
  misc_feature 1..22: note = Synthetic
  source 1..22: mol_type = other DNA
gtgaggtaagatggtggctaat

SEQ ID NO: 27 - DNA, 21 bp, synthetic construct
  misc_feature 1..21: note = Synthetic
  source 1..21: mol_type = other DNA
agctttgatccctgacatctg

SEQ ID NO: 28 - DNA, 22 bp, synthetic construct
  misc_feature 1..22: note = Synthetic
  source 1..22: mol_type = other DNA
tctaggtgaggtcttctcaagt